(19) United States
(12) Patent Application Publication
(10) Pub. No.: US 2025/0066431 A1
(43) Pub. Date: Feb. 27, 2025

(54) IDENTIFICATION OF CHANNELRHODOPSIN-2 (CHR2) MUTATIONS AND METHODS OF USE

(71) Applicant: Wayne State University, Detroit, MI (US)

(72) Inventor: Zhuo-Hua PAN, Detroit, MI (US)

(21) Appl. No.: 18/639,381

(22) Filed: Apr. 18, 2024

Related U.S. Application Data
(63) Continuation of application No. 17/202,064, filed on Mar. 15, 2021, now abandoned.
Application No. 17/202,064 is a continuation of application No. 14/383,211, filed on Sep. 5, 2014, now Pat. No. 10,947,281.
(60) Provisional application No. 61/606,663, filed on Mar. 5, 2012.

(51) Int. Cl. C07K 14/405; A61K 38/16; A61K 48/00; A61P 27/02; C07K 14/00

(52) U.S. Cl. CPC C07K 14/405; A61K 38/16; A61P 27/02; C07K 14/00; A61K 48/00

(57) ABSTRACT

The invention provides compositions and kits including at least one nucleic acid or polypeptide molecule encoding for a mutant ChR2 protein. Methods of the invention include administering a composition comprising a mutant ChR2 to a subject to preserve, improve, or restore phototransduction. Preferably, the compositions and methods of the invention are provided to a subject having impaired vision, thereby restoring vision to normal levels.

### CROSS-REFERENCE TO RELATED APPLICATIONS

This application is a continuation application of U.S. patent application Ser. No. 17/202,064, filed Mar. 15, 2021, which is a continuation of and claims the benefit of U.S. patent application Ser. No. 14/383,211 filed Sep. 5, 2014, now U.S. Pat. No. 10,947,281 issued Mar. 16, 2021, which is a national stage application filed under 35 U.S.C. § 371 of International Application No. PCT/US2013/029171 filed Mar. 5, 2013, which claims the benefit of and priority under 35 U.S.C. § 119 (e) to U.S. Provisional Application No. 61/606,663, filed Mar. 5, 2012, the contents of which are incorporated by reference herein in their entireties.

### GOVERNMENT SUPPORT

This invention was made with U.S. Government support under the National Institutes of Health/National Eye Institute grant NIH EY 17130. The Government has certain rights in the invention.

### INCORPORATION BY REFERENCE OF SEQUENCE LISTING

The present application contains a sequence listing which has been submitted in XML format. The content of the computer readable XML file named “6065_0506C2_SequenceListing.xml”, which was created on Apr. 18, 2024 and is 58.7 kilobytes in size, is hereby incorporated by reference in its entirety.

### FIELD OF THE INVENTION

This invention relates generally to the field of molecular biology. Mutations in the Channelopsin-2 (Chop2) gene are identified. Compositions comprising a mutant Chop2 gene are used in therapeutic methods to improve and restore vision loss.

### BACKGROUND OF THE INVENTION

The retina is composed of photoreceptors (or photoreceptor cells, rods and cones). Photoreceptors are highly specialized neurons that are responsible for phototransduction, or the conversion of light (in the form of electromagnetic radiation) into electrical and chemical signals that propagate a cascade of events within the visual system, ultimately generating a representation of our world.

Photoreceptor loss or degeneration severely compromises, if not completely inhibits, phototransduction of visual information within the retina. Loss of photoreceptor cells and/or loss of a photoreceptor cell function are the primary causes of diminished visual acuity, diminished light sensitivity, and blindness. There is a long-felt need in the art for compositions and method that restore photosensitivity of the retina of a subject experiencing vision loss.

### SUMMARY OF THE INVENTION

The invention provides a solution for the long-felt need for a method of restoring and/or increasing the light sensitivity of photoreceptor cells by expression of advantageous mutations, and/or combinations thereof, of the Channelopsin-2 (Chop2) gene, and subsequently providing methods for Channelopsin-2 (Chop2)-based gene therapy.

Channelopsin-2 (Chop2)-based gene therapy offers a superior strategy for restoring retinal photosensitivity after photoreceptor degeneration. The protein product of the Chop2 gene, when bound to the light-isomerizable chromophore all-trans-retinal, forms a functional light-gated channel, called channelrhodopsin-2 (ChR2). Native ChR2 shows low light sensitivity. Recently, two mutant ChR2s, L132C and T159C, were reported to markedly increase their light sensitivity (Kleinlogel et al. (2011) Nat Neurosci. 14:513-8; Berndt et al. (2011) Proc Natl Acad Sci USA. 108:7595-600; Prigge et al. (2012) J Biol Chem. 287 (38) 3104:12; the contents of each of which are incorporated herein in their entireties). The properties of these two ChR2 mutants (i.e., L132C and T159C) were examined and compared with a number of double mutants at these two sites to identify suitable candidates for therapeutic methods. Compositions comprising one or more of these mutations are provided to a subject in need thereof for the purpose of restoring vision. Specifically, desired mutations in the Chop2 gene are introduced to a cell and/or integrated into the genomic DNA of a cell to improve or restore vision. Desired mutations in the Chop2 gene that are introduced to a cell to improve or restore vision may also remain episomal, not having integrated into the genomic DNA.

Mutations at the L132 or T159 amino acid positions of Chop2 (and therefore, the resulting ChR2) markedly lower the threshold light intensity that is required to elicit the ChR2-mediated photocurrent. Double mutants at the amino acid positions L132 and T159 further increase the photocurrent at low light intensities, exceeding that of either of the corresponding single mutations. Retinal ganglion cells expressing the double mutants at the L132 and T159 positions can respond to light intensities that fall within the range of normal outdoor lighting conditions but should still maintain adequate, and high temporal resolution that are suitable for restoring useful vision. Thus, mutant Chop2 protein of the present invention that form mutant ChR2s having improved light sensitivity are used alone or in combination to restore or improve vision.

Specifically, the invention provides an isolated polypeptide molecule comprising or consisting of SEQ ID NO: 26 in which the amino acid at position 132 of SEQ ID NO: 26 is not leucine (L). In certain embodiments of the isolated polypeptide molecule, the amino acid at position 132 is cysteine (C) or alanine (A). When the amino acid at position 132 is cysteine (C), the polypeptide molecule may comprise or consist of SEQ ID NO: 13. When the amino acid at position 132 is alanine (A), the polypeptide molecule may comprise or consist of SEQ ID NO: 20.

The invention provides an isolated polypeptide molecule comprising or consisting of SEQ ID NO: 26 in which the amino acid at position 159 of SEQ ID NO: 26 is not a threonine (T). In certain embodiments of the isolated polypeptide molecule, the amino acid at position 159 is cysteine (C), serine (S), or alanine (A). When the amino acid at position 159 is cysteine (C), the polypeptide molecule may comprise or consist of SEQ ID NO: 14. When the amino acid at position 159 is serine (S), the polypeptide molecule may comprise or consist of SEQ ID NO: 17. When the amino acid at position 159 is alanine (A), the polypeptide molecule may comprise or consist of SEQ ID NO: 23.

The invention provides isolated polypeptide molecule comprising or consisting of SEQ ID NO: 26 in which the amino acid at position 132 of SEQ ID NO: 26 is not leucine (L) and the amino acid at position 159 is not threonine (T). In certain embodiments of the isolated polypeptide molecule comprising or consisting of SEQ ID NO: 26 in which the amino acid at position 132 of SEQ ID NO: 26 is not leucine (L) and the amino acid at position 159 is not threonine (T), the amino acid at position 132 is cysteine (C), and the amino acid at position 159 is cysteine (C). In a preferred embodiment of this isolated polypeptide molecule, the polypeptide molecule comprises or consists of SEQ ID NO: 16. The invention provides an isolated nucleic acid molecule that encodes for the isolated polypeptide comprising or consisting of SEQ ID NO: 16. Preferably, the isolated nucleic acid molecule that encodes for the isolated polypeptide comprising or consisting of SEQ ID NO: 16, is a nucleic acid molecule that comprises or consists of SEQ ID NO: 15.

In certain embodiments of the isolated polypeptide molecule comprising or consisting of SEQ ID NO: 26 in which the amino acid at position 132 of SEQ ID NO: 26 is not leucine (L) and the amino acid at position 159 is not threonine (T), the amino acid at position 132 is cysteine (C) and the amino acid at position 159 is serine (S). The isolated polypeptide molecule comprising or consisting of SEQ ID NO: 26 in which the amino acid at position 132 of SEQ ID NO: 26 is not leucine (L) and the amino acid at position 159 is not threonine (T), may comprise or consist of SEQ ID NO: 19. Alternatively, or in addition, the isolated polypeptide molecule comprising or consisting of SEQ ID NO: 26 in which the amino acid at position 132 of SEQ ID NO: 26 is not leucine (L) and the amino acid at position 159 is not threonine (T), wherein the amino acid at position 132 is cysteine (C) and wherein the amino acid at position 159 is serine (S) may comprise or consist of SEQ ID NO: 19. The invention provides an isolated nucleic acid molecule that encodes for the isolated polypeptide that comprises or consists of SEQ ID NO: 19. Preferably, the nucleic acid molecule comprises or consists of SEQ ID NO: 18.

In certain embodiments of the isolated polypeptide molecule comprising or consisting of SEQ ID NO: 26 in which the amino acid at position 132 of SEQ ID NO: 26 is not leucine (L) and the amino acid at position 159 is not threonine (T), the amino acid at position 132 is alanine (A) and the amino acid at position 159 is cysteine (C). The isolated polypeptide molecule comprising or consisting of SEQ ID NO: 26 in which the amino acid at position 132 of SEQ ID NO: 26 is not leucine (L) and the amino acid at position 159 is not threonine (T) may comprise or consist of SEQ ID NO: 22. Alternatively, or in addition, the isolated polypeptide molecule comprising or consisting of SEQ ID NO: 26 in which the amino acid at position 132 of SEQ ID NO: 26 is not leucine (L) and the amino acid at position 159 is not threonine (T), wherein the amino acid at position 132 is alanine (A) and wherein the amino acid at position 159 is cysteine (C) may comprise or consist of SEQ ID NO: 22. The invention provides an isolated nucleic acid molecule that encodes for the isolated polypeptide that comprises or consists of SEQ ID NO: 22. Preferably, this nucleic acid molecule comprises or consists of SEQ ID NO: 21.

In certain embodiments of the isolated polypeptide molecule comprising or consisting of SEQ ID NO: 26 in which the amino acid at position 132 of SEQ ID NO: 26 is not leucine (L) and the amino acid at position 159 is not threonine (T), the amino acid at position 132 is cysteine (C) and the amino acid at position 159 is alanine (A). The isolated polypeptide molecule comprising or consisting of SEQ ID NO: 26 in which the amino acid at position 132 of SEQ ID NO: 26 is not leucine (L) and the amino acid at position 159 is not threonine (T) may comprise or consist of SEQ ID NO: 25. Alternatively, or in addition, the isolated polypeptide molecule comprising or consisting of SEQ ID NO: 26 in which the amino acid at position 132 of SEQ ID NO: 26 is not leucine (L) and the amino acid at position 159 is not threonine (T), wherein the amino acid at position 132 is cysteine (C) and wherein the amino acid at position 159 is alanine (A) may comprise or consist of SEQ ID NO: 25. The invention provides an isolated nucleic acid molecule that encodes for the isolated polypeptide that comprises or consists of SEQ ID NO: 25. Preferably, this nucleic acid molecule comprises or consists of SEQ ID NO: 24.

The invention provides any one of the isolated polypeptide molecules described herein, wherein the polypeptide molecule encodes for a mutant Chop2 protein that forms a mutant ChR2, which elicits a current in response to a threshold intensity of light that is lower than the threshold of a wild type ChR2 protein. Moreover, the current conducts cations. Exemplary cations include, but are not limited to, H⁺, Na⁺, K⁺, and Ca²⁺ ions. The ChR2 wild type and mutant proteins described herein non-specifically conduct cations. Consequently, the current conducts one or more of the following: H⁺, Na⁺, K⁺, and Ca²⁺ ions.

The invention provides any one of the isolated polypeptide molecules described herein further comprising a pharmaceutically acceptable carrier. The invention also provides a composition comprising at least one isolated polynucleotide molecule described herein. The composition may further comprise a pharmaceutically-acceptable carrier.

The invention provides an isolated nucleic acid molecule that encodes for any of the isolated polypeptides described herein. Moreover, the isolated nucleic acid molecule may further include a pharmaceutically acceptable carrier. The invention also provides a composition comprising at least one isolated nucleic acid molecule described herein. The composition may further comprise a pharmaceutically-acceptable carrier.

The invention provides a cell, wherein the cell has been contacted with or comprises an isolated polypeptide molecule of the invention. Moreover, the invention provides a cell, wherein the cell has been contacted with or comprises an isolated nucleic acid molecule that encodes for an isolated polypeptide molecule of the invention. The invention provides, a composition comprising, consisting essentially of, or consisting of a cell that comprises an isolated polypeptide molecule of the invention or a nucleic acid molecule that encodes for an isolated polypeptide molecule of the invention. Cells of the invention may be contacted with the isolated polypeptide or an isolated nucleic acid encoding the polypeptide in vitro, ex vivo, in vivo, or in situ. In certain embodiments of the invention, the cell is a photoreceptor: a horizontal cell: a bipolar cell: an amacrine cell, and, especially, an AII amacrine cell; or a retinal ganglion cell, including a photosensitive retinal ganglion cell. Preferably, the cell is a retinal ganglion cell, a photosensitive retinal ganglion cell, a bipolar cell, an ON-type bipolar cell, a rod bipolar cell, or an AII amacrine cell. In certain aspects of the invention, the cell is a photoreceptor, a bipolar cell, a rod bipolar cell, an ON-type cone bipolar cell, a retinal ganglion cell, a photosensitive retinal ganglion cell, a horizontal cell, an amacrine cell, or an AII amacrine cell.

The invention provides a method of improving or restoring vision, comprising administering to a subject any one of the compositions described herein. The invention further provides a prophylactic method of preserving vision, comprising administering to a subject any one of the compositions described herein.

The methods described herein may also be applied to those subjects who are healthy, blind (in part or in total), and/or those subjects with retinal degeneration (characterized by a loss of rod and/or cone photoreceptor cells), but may be dependent upon the activity of photosensitive retinal ganglion cells for a determination of ambient light levels. For example, the methods described herein can be used to preserve, improve, or restore the activity of a photosensitive retinal ganglion cell that mediates the transduction of light information for synchronizing circadian rhythms to the 24-hour light/dark cycle, pupillary control and reflexes, and photic regulation of melatonin release.

In certain embodiments of the methods of the invention, the subject may have normal vision or impaired vision. Alternatively, or in addition, the subject may be at risk for developing an ocular disease that leads to impairment of vision. For example, the subject may have a family history of, ocular disease, including, macular degeneration and retinitis pigmentosa. The subject may be at risk for incurring an eye injury that causes damage to photosensitive cells in the retina. The subject may have a genetic marker or genetic/congenital condition that results in impaired vision, low vision, legal blindness, partial blindness, or complete blindness. Subjects may have a refractive defect that results in myopia (near-sightedness) or hyperopia (far-sightedness).

Compositions of methods of the invention may be administered to a subject either systemically or locally. A preferred route of local administration is intravitreal injection.

Other features and advantages of the invention will be apparent from and are encompassed by the following detailed description and claims.

### BRIEF DESCRIPTION OF THE FIGURES

FIG. 1A-B show representative recordings of the light-evoked currents from wild-type (WT) ChR2, L132C, L132C/T159C, and L132C/159S mutants in HEK cells for comparison of their light sensitivity (A). The light stimuli (photons/cm²·s at 460 nm) were generated by a xenon arc lamp and attenuated by neutral density filters: ND4.0 (2.8×10¹⁴), ND3.0 (1.4×10¹⁵), ND2.5 (4.8×10¹⁵); ND2.0 (1.6×10¹⁶), ND1.0 (1.3×10¹⁷), ND0 (1.2×10¹⁸). The same current traces in FIG. 1A are shown at a different current scale in FIG. 1B. The traces pointed by arrows are evoked by the same light intensity (ND2.5).

FIG. 2 shows representative recordings of the light-evoked currents from wild-type (WT) ChR2, T159C, L132C, L132C/T159C, and L132C/T159S mutants to a 10 ms light pulse (1.2×10¹⁸ photons/cm²/s at 460 nm) in HEK cells for comparison of their deactivation time course (decay time course after light off).

FIG. 3 shows representative multichannel array recordings of WT ChR2, L132C, L132C/T159C, and L132C/T159S mediated spiking activities from retinal ganglion cells in retinal whole-mounts for comparison of their light sensitivity. Light stimuli (photons/cm²/s) was generated by a 473 nm blue laser and attenuated by neutral density filters: ND0 (6.3×10¹⁶), ND1.0 (7.4×10¹⁵), ND1.5 (2.7×10¹⁵), ND2.0 (7.3×10¹⁴), ND2.5 (3.2×10¹⁴), ND3.0 (8.5×10¹³), ND3.5 (3.8×10¹³), and ND4.0 (9.5×10¹²).

FIG. 4A-B show representative multichannel array recordings of WT ChR2, L132C, L132C/T159C, and L132C/T159S mediated spiking activities from retinal ganglion cells in retinal whole-mounts for comparison of their temporal dynamics. In each panel, the raster plots of 10 consecutive light-elicited spikes originated from a single neuron (top) and the averaged spike rate histograms (bottom) are shown. Light pulses at different frequency was generated by a 473 nm blue laser with intensities about one log unit above the threshold intensity of each mutant. Recordings of WT ChR2 and L132C are shown in FIG. 4A, and recordings of L132C/T159C and L132C/T159S are shown in FIG. 4B.

### DETAILED DESCRIPTION

### Visual System

The central nervous system mediates vision (also referred to herein as sight) through specialized cells and unique methods of signal transduction present in the visual system. The principle responsibility of the visual system is to transform light, in the form of electromagnetic radiation, into a representation or image of the surrounding world. In addition to the “visual” function of this system, the visual system also regulates the pupillary light reflex (PLR), circadian photoentrainment to periodic light/dark cycles, and release of the hormone melatonin.

The cells of the retina are the first cells of the visual or nervous system to encounter light (electromagnetic radiation of varying wavelengths and intensities). Photons travel through the cornea, pupil, and lens before reaching the retina. The retina has a unique structure because the photoreceptor cells that directly absorb photons are located in the outer layer of the retina. Photons that traverse the lens first encounter an inner layer of retinal ganglion cells (a minority of which are photosensitive through the expression of the opsin, melanopsin) and an intermediate layer of bipolar cells before reaching the outer layer of photoreceptor cells (also known as rods and cones). Rod photoreceptors operate in dim illumination condition (scotopic vision) while cone photoreceptors operate in bright illumination conditions (photopic vision) responsible for color vision. Cone photoreceptors synapse directly onto ON- and OFF-type cone bipolar cells, which in turn, synapse directly onto ON- and OFF-type retinal ganglion cells. Rod photoreceptors synapse to rod bipolar cells (a unique type of bipolar cells, which is ON-type), which synapse to AII amacrine cells. The AII amacrine cells then relay the visual signals to ON-type cone bipolar cells through gap junction and to OFF-type cone bipolar cells as well as OFF ganglion cells through inhibitory glycinergic synapses. Retinal ganglion cells are responsible for relating visual information to neurons of the brain.

### Phototransduction

Within the retina, photoreceptor cells absorb photon particles and transform the raw data of light frequency and wavelength into chemical and subsequently electrical signals that propagate this initial information throughout the visual and nervous systems. Specifically, an opsin protein located on the surface of a photoreceptor (rod, cone, and/or photosensitive retinal ganglion cell) absorbs a photon and initiates an intracellular signaling cascade, which results in the hyperpolarization of the photoreceptor. In the dark, the opsin proteins absorb no photons, the photoreceptors are depolarized. The visual signals of photoreceptors then relay through bipolar cells, amacrine cells, and ganglion cells to the high visual centers in the brain. Specifically, when rod and cone photoreceptors are depolarized (in the dark), they cause the depolarization of rod bipolar cells and ON-type cone bipolar cells, but the hyperpolarization of OFF-type cone bipolar cells, which in turn cause the depolarization of AII amacrine cells and the increase of the spiking of ON-type retinal ganglion cells and the decrease of the spiking of OFF-type retinal ganglion cells. The opposite happens (to rod, ON- and OFF-bipolar cells, AII amacrine and ON- and OFF-ganglion cells), when rod and cone photoreceptors are hyperpolarized (in response to light).

Light information is processed and refined significantly by the actions of photoreceptors, bipolar cells, horizontal cells, amacrine cells, and retinal ganglion cells. To add to the complexity of this system, photoreceptors are found in three main varieties, including rods, cones (of which three types respond most strongly to distinct wavelengths of light), and photosensitive retinal ganglion cells. Thus, a first layer of information processing occurs at the level of the photoreceptors which respond differentially to certain wavelengths and intensities of light. Bipolar cells of the retina receive information from both photoreceptor cells and horizontal cells. Horizontal cells of the retina receive information from multiple photoreceptor cells, and, therefore, integrate information between cell types and across distances in the retina. Bipolar cells further integrate information directly from photoreceptor cells and horizontal cells by producing mainly graded potentials to retinal ganglion cells, although some recent studies indicate that some bipolar cells can generate action potentials. Cone bipolar cells synapse on retinal ganglion cells and amacrine cells while rod bipolar cells synapse only to AII amacrine cells. Similar to horizontal cells, most amacrine cells integrate information laterally within the retina. Unlike horizontal cells, most amacrine cells are inhibitory (GABAergic) interneurons. Amacrine cells are also more specialized than horizontal cells, because each amacrine cell specifically synapses on a particular type of bipolar cell (one of the ten varieties of bipolar cell). Particularly, the AII amacrine cell is a critical relay neuron in the rod pathway (under scotopic vision when cone photoreceptors do not respond). The AII amacrine cells receive synaptic inputs from rod bipolar cells and then piggy-back the signals to cone pathway through ON- and OFF-cone bipolar cells to ON- and OFF-ganglion cells as described above. Therefore, expression of Chop2, and the resulting formation of ChR2, in rod bipolar cells or AII amacrine cells can create both ON and OFF responses in retinal ganglion cells. Furthermore, retinal ganglion cells integrate information from bipolar cells and from amacrine cells. Although retinal ganglion cells vary significantly with respect to size, connectivity, and responses to visual stimulation (e.g. visual fields), all retinal ganglion cells extend a long axon into the brain. Except for a minute portion of the retinal ganglion cells that transduce non-visual information regarding the pupillary light reflex and circadian entrainment, the totality of axons extending from the retinal ganglion cells form the optic nerve, optic chiasm, and optic tract of the central nervous system. Consequently, a significant amount of information processing occurs in the retina itself.

Photoreceptor cells express endogenous opsin proteins, such as rhodopsin. The mutant Chop2 proteins of the invention may be expressed in any cell type, and form functional ChR2 channels. Preferably, the cell is a retinal cell. Exemplary cells, include, but are not limited to, photoreceptor cells (e.g., rods, cones, and photosensitive retinal ganglion cells), horizontal cells, bipolar cells, amacrine cells, and retinal ganglion cells.

### Channelopsin-2 (Chop2)

Channelopsin-2 (Chop2) was first isolated from the green algae, Chlamydomonas reinhardtii. Channelopsin-2 is a seven transmembrane domain protein that becomes photo-switchable (light sensitive) when bound to the chromophore all-trans-retinal. Chop2, when linked to a retinal molecule via Schiff base linkage forms a light-gated, nonspecific, inwardly rectifying, cation channel, called Channelrhodopsin-2 (Chop2 retinalidene, abbreviated ChR2).

As referred to herein, “channelopsin-2” or “Chop2” refers to the gene that encodes channelopsin-2, which then forms Channelrhodopsin-2 (ChR2) once bound to retinal. Gene constructs of the present invention refer primarily to channelopsin-2 (i.e., without the retinal), and all Chop2 variants disclosed herein form functional channelrhodopsin-2 variants. The methods disclosed herein may include delivering Chop2 to cells without exogenous retinal. It is understood that upon expression of Chop2 in cells (i.e., retinal neurons), endogenously available retinal binds to the wild-type Chop2 or the Chop2 mutants of the present invention to form functional light-gated channels, WT ChR2 or mutant ChR2. As such, Chop2 proteins, as referred to herein, can also be synonymous with ChR2.

As used herein, “channelrhodopsin-2” or “ChR2” refers to the retinal-bound functional light-sensitive channel. In one embodiment, the bound retinal may be provided exogenously. In a preferred embodiment, the bound retinal is provided from endogenous levels available in the cell. The present invention also encompasses the functional channelrhodopsin-2 channels formed by the polypeptides and polynucleotides encoding the Chop2 mutants described herein.

Upon illumination by the preferred dose of light radiation, ChR2 opens the pore of the channel, through which H⁺, Na⁺, K⁺, and/or Ca²⁺ ions flow into the cell from the extracellular space. Activation of the ChR2 channel typically causes a depolarization of the cell expressing the channel. Depolarized cells produce graded potentials and or action potentials to carry information from the Chop2/ChR2-expressing cell to other cells of the retina or brain.

The wild type form of ChR2 or mutant ChR2s with high temporal resolution have become a central focus of neuroscience research. When expressed in a mammalian neuron, ChR2 mediates light-controlled depolarization of in vitro or ex vivo cultures. Wild type ChR2s or mutant ChR2s with high temporal resolution (the latter usually display low light sensitivity) presents several challenges that must be addressed to enable their use for the purpose of vision restoration. For the purpose of vision restoration, the ChR2 with high light sensitivity rather than high temporal resolution is desired.

Wild type ChR2 proteins require illumination from high blue light intensities for full activation (i.e. 10¹⁸-10¹⁹ photons s⁻¹ cm⁻² at a wavelength of 480 nm). Continuous illumination of this type can damage cells.

The kinetics of the wild type ChR2 protein is suboptimal for maximizing channel efficacy. Efficacy can be increased by modifying one or more amino acids of the wild type ChR2 protein either to prolong the open state of the channel or increase the unit conductance of the channel, or both. The single-channel conductance of wild-type ChR2 is small. Thus, neuronal activation in vivo would either require high expression of the wild type channel or very intense activation with the preferred wavelength of blue-light. A simpler solution may be found by altering the channel conductance or to prolong the channel open time. Either one of these mechanisms and, in particular, the combination of these mechanisms, enable lower and safer light intensities to be used to achieve the same level of cellular depolarization.

For example, mutant ChR2 proteins of the invention achieve greater light sensitivity through the prolongation of the channel open state. Consequently, each mutant ChR2 channel conducts a greater photocurrent than a wild type ChR2 channel when activated by the same light intensities. Therefore, the mutant channels are activated by light intensities that are lower than those required for activation of the wild type ChR2 channels. Quantitatively, detectable spiking activity of retinal ganglion cells expressing mutant ChR2 proteins can be elicited by a light intensity that is 1.5-2 log units lower than the light intensity required to elicit spiking activity from retinal ganglion cells expressing wild type ChR2. Thus, the light intensities required to activate the mutant ChR2 proteins are close to or fall within the range of normal outdoor lighting conditions.

The following sequences provide non-limiting examples of wild type and mutant Chop2 proteins, and polynucleotides encoding said WT and mutant Chop2 proteins of the invention, and forming WT and mutant ChR2s of the invention.

A wild type (WT) Chop2 of the invention may be encoded by the following Chlamydomonas reinhardtii chlamyopsin 4 light-gated ion channel (COP4) mRNA sequence (GenBank Accession No. XM_001701673, and SEQ ID NO: 1):

| | |
|---|---|
| 1 | gcagcaccat acttgacatc tgtcgccaag caagcattaa acatggatta tggaggcgcc |
| 61 | ctgagtgccg ttgggcgcga gctgctattt gtaacgaacc cagtagtcgt caatggctct |
| 121 | gtacttgtgc ctgaggacca gtgttactgc gcgggctgga ttgagtcgcg tggcacaaac |
| 181 | ggtgcccaaa cggcgtcgaa cgtgctgcaa tggcttgctg ctggcttctc catcctactg |
| 241 | cttatgtttt acgcctacca aacatggaag tcaacctgcg gctgggagga gatctatgtg |
| 301 | tgcgctatcg agatggtcaa ggtgattctc gagttcttct tcgagtttaa gaacccgtcc |
| 361 | atgctgtatc tagccacagg ccaccgcgtc cagtggttgc gttacgccga gtggcttctc |
| 421 | acctgcccgg tcattctcat tcacctgtca aacctgacgg gcttgtccaa cgactacagc |
| 481 | aggcgcacca tgggtctgct tgtgtctgat attggcacaa ttgtgtgggg cgccacttcc |
| 541 | gccatggcca ccggatacgt caaggtcatc ttcttctgcc tgggtctgtg ttatggtgct |
| 601 | aacacgttct ttcacgctgc caaggcctac atcgagggtt accacaccgt gccgaagggc |
| 661 | cggtgtcgcc aggtggtgac tggcatggct tggctcttct tcgtatcatg gggtatgttc |
| 721 | cccatcctgt tcatcctcgg ccccgagggc ttcggcgtcc tgagcgtgta cggctccacc |
| 781 | gtcggccaca ccatcattga cctgatgtcg aagaactgct ggggtctgct cggccactac |
| 841 | ctgcgcgtgc tgatccacga gcatatcctc atccacggcg acattcgcaa gaccaccaaa |
| 901 | ttgaacattg gtggcactga gattgaggtc gagacgctgg tggaggacga ggccgaggct |
| 961 | ggcgcggtca acaagggcac cggcaagtac gcctcccgcg agtccttcct ggtcatgcgc |
| 1021 | gacaagatga aggagaaggg cattgacgtg cgcgcctctc tggacaacag caaggaggtg |
| 1081 | gagcaggagc aggccgccag ggctgccatg atgatgatga acggcaatgg catgggtatg |
| 1141 | ggaatgggaa tgaacggcat gaacggaatg ggcggtatga acgggatggc tggcggcgcc |
| 1201 | aagcccggcc tggagctcac tccgcagcta cagcccggcc gcgtcatcct ggcggtgccg |
| 1261 | gacatcagca tggttgactt cttccgcgag cagtttgctc agctatcggt gacgtacgag |
| 1321 | ctggtgccgg ccctgggcgc tgacaacaca ctggcgctgg ttacgcaggc gcagaacctg |
| 1381 | ggcggcgtgg actttgtgtt gattcacccc gagttcctgc gcgaccgctc tagcaccagc |
| 1441 | atcctgagcc gcctgcgcgg cgcgggccag cgtgtggctg cgttcggctg ggcgcagctg |
| 1501 | gggcccatgc gtgacctgat cgagtccgca aacctggacg gctggctgga gggcccctcg |
| 1561 | ttcggacagg gcatcctgcc ggcccacatc gttgccctgg tggccaagat gcagcagatg |
| 1621 | cgcaagatgc agcagatgca gcagattggc atgatgaccg gcggcatgaa cggcatgggc |
| 1681 | ggcggtatgg gcggcggcat gaacggcatg ggcggcggca acggcatgaa caacatgggc |
| 1741 | aacggcatgg gcggcggcat gggcaacggc atgggcggca atggcatgaa cggaatgggt |
| 1801 | ggcggcaacg gcatgaacaa catgggcggc aacggaatgg ccggcaacgg aatgggcggc |
| 1861 | ggcatgggcg gcaacggtat gggtggctcc atgaacggca tgagctccgg cgtggtggcc |
| 1921 | aacgtgacgc cctccgccgc cggcggcatg ggcggcatga tgaacggcgg catggctgcg |
| 1981 | ccccagtcgc ccggcatgaa cggcggccgc ctgggtacca acccgctctt caacgccgcg |
| 2041 | ccctcaccgc tcagctcgca gctcggtgcc gaggcaggca tgggcagcat gggaggcatg |
| 2101 | ggcggaatga gcggaatggg aggcatgggt ggaatggggg gcatgggcgg cgccggcgcc |
| 2161 | gccacgacgc aggctgcggg cggcaacgcg gaggcggaga tgctgcagaa tctcatgaac |
| 2221 | gagatcaatc gcctgaagcg cgagcttggc gagtaaaagg ctggaggccg gtactgcgat |
| 2281 | acctgcgagc tcgcgcgcct gactcgtcgt acacacggct caggagcacg cgcgcgtgga |
| 2341 | cttctcaacc tgtgtgcaac gtatctagag cggcctgtgc gcgaccgtcc gtgagcattc |
| 2401 | cggtgcgatc ttcccgcctt cgcaccgcaa gttcccttcc tggccctgct gcgcctgacg |
| 2461 | catcgtccga acggaagggc ggcttgatca gtaaagcatt gaagactgaa gtcgtgcgac |
| 2521 | cgtagtgcta tggctctgca cgtaagtggg cgctgccctg cttactacgc attgcccaag |
| 2581 | actgcttcct tttggtggcc gaggccctgg tcccacatca ttcatttgca taacgtactg |
| 2641 | tttagttaca tacgctttgc ttaacctcga caattgcaac atgggctgag agtccgtacg |
| 2701 | gcggctatgg acgaaggtgt tatcggatgt gattaggaat ctcggttgaa aggcttcgag |
| 2761 | aaagtgagct tcatctgtgg cttctgttgg ggtcatcaag aagaacgacg gtaaggcaaa |
| 2821 | cgaggtaaaa gtggcacgtc tttgtgcaca acgggcccgt ggagagtggg ggagtgcatg |
| 2881 | tgtgcggtcc taacacgcga gtgcaaagcg ggcttttctg gagctgggtt acggtctggc |
| 2941 | tcggcaactg ctctgtgttt taaccacagc ttcggaagtc tgggtatgtt ttgttggcag |
| 3001 | aaacatttgg gtaacttgag ggtgattcgt ctggagtcgg acaacatggc tgccgtccgt |
| 3061 | gtgcagggac ggtaatcaat gagctggagc tgtgatgctc accacacgtt gcatacccct |
| 3121 | gcttacaaaa acactttgat gtcgtggcca aactatgcgt gagcaaagag ttaaagaggc |
| 3181 | atgagtgcat ggttgcggac gtgcgcaaca attgcatcaa gtatttgacg ccttcaagcc |
| 3241 | aacaagtgcg cgcgcggcaa cttgattaac acgccggacg cagtggtggg ggcgtgtaca |
| 3301 | gtgtttatga gctgccattc tgcgatccgt agtgttaggt tgcgtgtgac gccgcgcggc |
| 3361 | tgtgggccct tacatggaga gttgggtgct tcaccacacg gttggcgccg ctgaagggtg |
| 3421 | tgctatgttt tggtaaagcc ggggccctga agaccgcaac cgtagaaccg tactgaaagg |
| 3481 | gtgtcagccc ggggtaactg gatgccctgg gacatagcta ttaatgttga agtgaagccg |
| 3541 | tcaagccgag tgccgtgcgc cgctgtatca ccaaggcccg tccta |

A wild type (WT) ChR2 of the invention may be encoded by the following Chlamydomonas reinhardtii chlamyopsin 4 light-gated ion channel (COP4) amino acid sequence (GenBank Accession No. XP_001701725, and SEQ ID NO: 2):

| | |
|---|---|
| 1 | mdyggalsav grellfvtnp vvvngsvlvp edqcycagwi esrgtngaqt asnvlqwlaa |
| 61 | gfsilllmfy ayqtwkstcg weeiyvcaie mvkvilefff efknpsmlyl atghrvqwlr |
| 121 | yaewlltcpv ilihlsnltg lsndysrrtm gllvsdigti vwgatsamat gyvkviffcl |
| 181 | glcygantff haakayiegy htvpkgrcrq vvtgmawlff vswgmfpilf ilgpegfgvl |
| 241 | svygstvght iidlmskncw gllghylrvl ihehilihgd irkttklnig gteievetlv |
| 301 | edeaeagavn kgtgkyasre sflvmrdkmk ekgidvrasl dnskeveqeq aaraammmmn |
| 361 | gngmgmgmgm ngmngmggmn gmaggakpgl eltpqlqpgr vilavpdism vdffreqfaq |
| 421 | lsvtyelvpa lgadntlalv tqaqnlggvd fvlihpeflr drsstsilsr lrgagqrvaa |
| 481 | fgwaqlgpmr dliesanldg wlegpsfgqg ilpahivalv akmqqmrkmq qmqqigmmtg |
| 541 | gmngmgggmg ggmngmgggn gmnnmgngmg ggmgngmggn gmngmgggng mnnmggngma |
| 601 | gngmgggmgg ngmggsmngm ssgvvanvtp saaggmggmm nggmaapqsp gmnggrlgtn |
| 661 | plfnaapspl ssqlgaeagm gsmggmggms gmggmggmgg mggagaattq aaggnaeaem |
| 721 | lqnlmneinr lkrelge |

A wild type (WT) Chop2 of the invention may be encoded by the following Chlamydomonas reinhardtii retinal binding protein (cop4) gene sequence (GenBank Accession No. AF461397, and SEQ ID NO: 3):

| | |
|---|---|
| 1 | gcatctgtcg ccaagcaagc attaaacatg gattatggag gcgccctgag tgccgttggg |
| 61 | cgcgagctgc tatttgtaac gaacccagta gtcgtcaatg gctctgtact tgtgcctgag |
| 121 | gaccagtgtt actgcgcggg ctggattgag tcgcgtggca caaacggtgc ccaaacggcg |
| 181 | tcgaacgtgc tgcaatggct tgctgctggc ttctccatcc tactgcttat gttttacgcc |
| 241 | taccaaacat ggaagtcaac ctgcggctgg gaggagatct atgtgtgcgc tatcgagatg |
| 301 | gtcaaggtga ttctcgagtt cttcttcgag tttaagaacc cgtccatgct gtatctagcc |
| 361 | acaggccacc gcgtccagtg gttgcgttac gccgagtggc ttctcacctg cccggtcatt |
| 421 | ctcattcacc tgtcaaacct gacgggcttg tccaacgact acagcaggcg caccatgggt |
| 481 | ctgcttgtgt ctgatattgg cacaattgtg tggggcgcca cttccgccat ggccaccgga |
| 541 | tacgtcaagg tcatcttctt ctgcctgggt ctgtgttatg gtgctaacac gttctttcac |
| 601 | gctgccaagg cctacatcga gggttaccac accgtgccga agggccggtg tcgccaggtg |
| 661 | gtgactggca tggcttggct cttcttcgta tcatggggta tgttccccat cctgttcatc |
| 721 | ctcggccccg agggcttcgg cgtcctgagc gtgtacggct ccaccgtcgg ccacaccatc |
| 781 | attgacctga tgtcgaagaa ctgctggggt ctgctcggcc actacctgcg cgtgctgatc |
| 841 | cacgagcata tcctcatcca cggcgacatt cgcaagacca ccaaattgaa cattggtggc |
| 901 | actgagattg aggtcgagac gctggtggag gacgaggccg aggctggcgc ggtcaacaag |
| 961 | ggcaccggca agtacgcctc ccgcgagtcc ttcctggtca tgcgcgacaa gatgaaggag |
| 1021 | aagggcattg acgtgcgcgc ctctctggac aacagcaagg aggtggagca ggagcaggcc |
| 1081 | gccagggctg ccatgatgat gatgaacggc aatggcatgg gtatgggaat gggaatgaac |
| 1141 | ggcatgaacg gaatgggcgg tatgaacggg atggctggcg gcgccaagcc cggcctggag |
| 1201 | ctcactccgc agctacagcc cggccgcgtc atcctggcgg tgccggacat cagcatggtt |
| 1261 | gacttcttcc gcgagcagtt tgctcagcta tcggtgacgt acgagctggt gccggccctg |
| 1321 | ggcgctgaca acacactggc gctggttacg caggcgcaga acctgggcgg cgtggacttt |
| 1381 | gtgttgattc accccgagtt cctgcgcgac cgctctagca ccagcatcct gagccgcctg |
| 1441 | cgcggcgcgg gccagcgtgt ggctgcgttc ggctgggcgc agctggggcc catgcgtgac |
| 1501 | ctgatcgagt ccgcaaacct ggacggctgg ctggagggcc cctcgttcgg acagggcatc |
| 1561 | ctgccggccc acatcgttgc cctggtggcc aagatgcagc agatgcgcaa gatgcagcag |
| 1621 | atgcagcaga ttggcatgat gaccggcggc atgaacggca tgggcggcgg tatgggcggc |
| 1681 | ggcatgaacg gcatgggcgg cggcaacggc atgaacaaca tgggcaacgg catgggcggc |
| 1741 | ggcatgggca acggcatggg cggcaatggc atgaacggaa tgggtggcgg caacggcatg |
| 1801 | aacaacatgg gcggcaacgg aatggccggc aacggaatgg gcggcggcat gggcggcaac |
| 1861 | ggtatgggtg gctccatgaa cggcatgagc tccggcgtgg tggccaacgt gacgccctcc |
| 1921 | gccgccggcg gcatgggcgg catgatgaac ggcggcatgg ctgcgcccca gtcgcccggc |
| 1981 | atgaacggcg gccgcctggg taccaacccg ctcttcaacg ccgcgccctc accgctcagc |
| 2041 | tcgcagctcg gtgccgaggc aggcatgggc agcatgggag gcatgggcgg aatgagcgga |
| 2101 | atgggaggca tgggtggaat ggggggcatg ggcggcgccg gcgccgccac gacgcaggct |
| 2161 | gcgggcggca acgcggaggc ggagatgctg cagaatctca tgaacgagat caatcgcctg |
| 2221 | aagcgcgagc ttggcgagta a |

A wild type (WT) Chop2 of the invention may be encoded by the following Chlamydomonas reinhardtii retinal binding protein (cop4) amino acid sequence (GenBank Accession No. AAM15777, and SEQ ID NO: 4):

| | |
|---|---|
| 1 | mdyggalsav grellfvtnp vvvngsvlvp edqcycagwi esrgtngaqt asnvlqwlaa |
| 61 | gfsilllmfy ayqtwkstcg weeiyvcaie mvkvilefff efknpsmlyl atghrvqwlr |
| 121 | yaewlltcpv ilihlsnltg lsndysrrtm gllvsdigti vwgatsamat gyvkviffcl |
| 181 | glcygantff haakayiegy htvpkgrcrq vvtgmawlff vswgmfpilf ilgpegfgvl |
| 241 | svygstvght iidlmskncw gllghylrvl ihehilihgd irkttklnig gteievetiv |
| 301 | edeaeagavn kgtgkyasre sflvmrdkmk ekgidvrasl dnskeveqeq aaraammmmn |
| 361 | gngmgmgmgm ngmngmggmn gmaggakpgl eltpqlqpgr vilavpdism vdffreqfaq |
| 421 | lsvtyelvpa lgadntlalv tqaqnlggvd fvlihpeflr drsstsilsr lrgagqrvaa |
| 481 | fgwaqlgpmr dliesanldg wlegpsfgqg ilpahivalv akmqqmrkmq qmqqigmmtg |
| 541 | gmngmgggmg ggmngmgggn gmnnmgngmg ggmgngmggn gmngmgggng mnnmggngma |
| 601 | gngmgggmgg ngmggsmngm ssgvvanvtp saaggmggmm nggmaapqsp gmnggrlgtn |
| 661 | plfnaapspl ssqlgaeagm gsmggmggms gmggmggmgg mggagaattq aaggnaeaem |
| 721 | lqnlmneinr lkrelge |

A wild type (WT) Chop2 of the invention may be encoded by the following Chlamydomonas reinhardtii sensory opsin B (CSOB) mRNA sequence (GenBank Accession No. AF508966, and SEQ ID NO: 5):

| | |
|---|---|
| 1 | ttgacatctg tcgccaagca agcattaaac atggattatg gaggcgccct gagtgccgtt |
| 61 | gggcgcgagc tgctatttgt aacgaaccca gtagtcgtca atggctctgt acttgtgcct |
| 121 | gaggaccagt gttactgcgc gggctggatt gagtcgcgtg gcacaaacgg tgcccaaacg |
| 181 | gcgtcgaacg tgctgcaatg gcttgctgct ggcttctcca tcctactgct tatgttttac |
| 241 | gcctaccaaa catggaagtc aacctgcggc tgggaggaga tctatgtgtg cgctatcgag |
| 301 | atggtcaagg tgattctcga gttcttcttc gagtttaaga acccgtccat gctgtatcta |
| 361 | gccacaggcc accgcgtcca gtggttgcgt tacgccgagt ggcttctcac ctgcccggtc |
| 421 | attctcattc acctgtcaaa cctgacgggc ttgtccaacg actacagcag gcgcaccatg |
| 481 | ggtctgcttg tgtctgatat tggcacaatt gtgtggggcg ccacttccgc catggccacc |
| 541 | ggatacgtca aggtcatctt cttctgcctg ggtctgtgtt atggtgctaa cacgttcttt |
| 601 | cacgctgcca aggcctacat cgagggttac cacaccgtgc cgaagggccg gtgtcgccag |
| 661 | gtggtgactg gcatggcttg gctcttcttc gtatcatggg gtatgttccc catcctgttc |
| 721 | atcctcggcc ccgagggctt cggcgtcctg agcgtgtacg gctccaccgt cggccacacc |
| 781 | atcattgacc tgatgtcgaa gaactgctgg ggtctgctcg gccactacct gcgcgtgctg |
| 841 | atccacgagc atatcctcat ccacggcgac attcgcaaga ccaccaaatt gaacattggt |
| 901 | ggcactgaga ttgaggtcga gacgctggtg gaggacgagg ccgaggctgg cgcggtcaac |
| 961 | aagggcaccg gcaagtacgc ctcccgcgag tccttcctgg tcatgcgcga caagatgaag |
| 1021 | gagaagggca ttgacgtgcg cgcctctctg gacaacagca aggaggtgga gcaggagcag |
| 1081 | gccgccaggg ctgccatgat gatgatgaac ggcaatggca tgggtatggg aatgggaatg |
| 1141 | aacggcatga acggaatggg cggtatgaac gggatggctg gcggcgccaa gcccggcctg |
| 1201 | gagctcactc cgcagctaca gcccggccgc gtcatcctgg cggtgccgga catcagcatg |
| 1261 | gttgacttct tccgcgagca gtttgctcag ctatcggtga cgtacgagct ggtgccggcc |
| 1321 | ctgggcgctg acaacacact ggcgctggtt acgcaggcgc agaacctggg cggcgtggac |
| 1381 | tttgtgttga ttcaccccga gttcctgcgc gaccgctcta gcaccagcat cctgagccgc |
| 1441 | ctgcgcggcg cgggccagcg tgtggctgcg ttcggctggg cgcagctggg gcccatgcgt |
| 1501 | gacctgatcg agtccgcaaa cctggacggc tggctggagg gcccctcgtt cggacagggc |
| 1561 | atcctgccgg cccacatcgt tgccctggtg gccaagatgc agcagatgcg caagatgcag |
| 1621 | cagatgcagc agattggcat gatgaccggc ggcatgaacg gcatgggcgg cggtatgggc |
| 1681 | ggcggcatga acggcatggg cggcggcaac ggcatgaaca acatgggcaa cggcatgggc |
| 1741 | ggcggcatgg gcaacggcat gggcggcaat ggcatgaacg gaatgggtgg cggcaacggc |
| 1801 | atgaacaaca tgggcggcaa cggaatggcc ggcaacggaa tgggcggcgg catgggcggc |
| 1861 | aacggtatgg gtggctccat gaacggcatg agctccggcg tggtggccaa cgtgacgccc |
| 1921 | tccgccgccg gcggcatggg cggcatgatg aacggcggca tggctgcgcc ccagtcgccc |
| 1981 | ggcatgaacg gcggccgcct gggtaccaac ccgctcttca acgccgcgcc ctcaccgctc |
| 2041 | agctcgcagc tcggtgccga ggcaggcatg ggcagcatgg gaggcatggg cggaatgagc |
| 2101 | ggaatgggag gcatgggtgg aatggggggc atgggcggcg ccggcgccgc cacgacgcag |
| 2161 | gctgcgggcg gcaacgcgga ggcggagatg ctgcagaatc tcatgaacga gatcaatcgc |
| 2221 | ctgaagcgcg agcttggcga gtaaaaggct ggaggccggt actgcgatac ctgcgagctc |
| 2281 | gcgcgcctga ctcgtcgtac acacggctca ggagcacgcg cgcgtggact tctcaacctg |
| 2341 | tgtgcaacgt atctagagcg gcctgtgcgc gaccgtccgt gagcattccg gtgcgatctt |
| 2401 | cccgccttcg caccgcaagt tcccttcctg gccctgctgc gcctgacgca tcgtccgaac |
| 2461 | ggaagggcgg cttgatcagt aaagcattga agactgaagt cgtgcgaccg tagtgctatg |
| 2521 | gctctgcacg taagtgggcg ctgccctgct tactacgcat tgcccaagac tgcttccttt |
| 2581 | tggtggccga ggccctggtc ccacatcatt catttgcata acgtactgtt tagttacata |
| 2641 | cgctttgctt aacctcgaca attgcaacat gggctgagag tccgtacggc ggctatggac |
| 2701 | gaaggtgtta tcggatgtga ttaggaatct cggttgaaag gcttcgagaa agtgagcttc |
| 2761 | ttctgtggct tctgttgggg tcatcaagaa gaacgacggt aaggcaaacg aggtaaaagt |
| 2821 | ggcacgtctt tgtgcacaac gggcccgtgg agagtggggg agtgcatgtg tgcggtccta |
| 2881 | acacgcgagt gcaaagcggg cttttctgga gctgggttac ggtctggctc ggcaactgct |
| 2941 | ctgtgtttta accacagctt cggaagtctg ggtatgtttt gttggcagaa acatttgggt |
| 3001 | aacttgaggg tgattcgtct ggagtcggac aacatggctg ccgtccgtgt gcagggacgg |
| 3061 | taatcaatga agctgaagct gtgatgctca ccacacgttg catacccctg cttacaaaaa |
| 3121 | cactttgatg tcgtggccaa actatgcgtg agcaaagagt taaagaggca tgagtgcatg |
| 3181 | gttgcggacg tgcgcaacaa ttgcatcaag tatttgacgc cttcaagcca acaagtgcgc |
| 3241 | gcgcggcaac ttgattaaca cgccggacgc agtggtgggg gcgtgtacag tgtttatgag |
| 3301 | ctgccattct gcgatccgta gtgttaggtt gcgtgtgacg ccgcgcggct gtgggccctt |
| 3361 | acatggagag ttgggtgctt caccacacgg ttggcgccgc tgaagggtgt gctatgtttt |
| 3421 | ggtaaagccg gggccctgaa gaccgcaacc gtagaaccgt actgaaaggg tgtcagcccg |
| 3481 | gggtaactgg atgccctggg acatagctat taatgttgaa gtgaagccgt caagccgagt |
| 3541 | gccgtgcgcc gctgtatcac caaggcccgt ccaaaaaaaa aaaaaaaaaa aaaaaaaaa |

A wild type (WT) Chop2 of the invention may be encoded by the following Chlamydomonas reinhardtii sensory opsin B (CSOB) amino acid sequence (GenBank Accession No. AAM44040, and SEQ ID NO: 6):

| | |
|---|---|
| 61 | gfsilllmfy ayqtwkstcg weeiyvcaie mvkvilefff efknpsmlyl atghrvqwlr |
| 121 | yaewlltcpv ilihlsnltg lsndysrrtm gllvsdigti vwgatsamat gyvkviffcl |
| 181 | glcygantff haakayiegy htvpkgrcrq vvtgmawlff vswgmfpilf ilgpegfgvl |
| 241 | svygstvght iidlmskncw gllghylrvl ihehilihgd irkttklnig gteievetlv |
| 301 | edeaeagavn kgtgkyasre sflvmrdkmk ekgidvrasl dnskeveqeq aaraammmmn |
| 361 | gngmgmgmgm ngmngmggmn gmaggakpgl eltpqlqpgr vilavpdism vdffreqfaq |
| 421 | lsvtyelvpa lgadntlalv tqaqnlggvd fvlihpeflr drsstsilsr lrgagqrvaa |
| 481 | fgwaqlgpmr dliesanldg wlegpsfgqg ilpahivalv akmqqmrkmq qmqqigmmtg |
| 541 | gmngmgggmg ggmngmgggn gmnnmgngmg ggmgngmggn gmngmgggng mnnmggngma |
| 601 | gngmgggmgg ngmggsmngm ssgvvanvtp saaggmggmm nggmaapqsp gmnggrlgtn |
| 661 | plfnaapspl ssqlgaeagm gsmggmggms gmggmggmgg mggagaattq aaggnaeaem |
| 721 | lqnlmneinr lkrelge |

A wild type (WT) Chop2 of the invention may be encoded by the following Chlamydomonas reinhardtii acop2 mRNA for archaeal-type opsin 2 nucleic acid sequence (GenBank Accession No. AB058891, and SEQ ID NO: 7):

| | |
|---|---|
| 1 | catctgtcgc caagcaagca ttaaacatgg attatggagg cgccctgagt gccgttgggc |
| 61 | gcgagctgct atttgtaacg aacccagtag tcgtcaatgg ctctgtactt gtgcctgagg |
| 121 | accagtgtta ctgcgcgggc tggattgagt cgcgtggcac aaacggtgcc caaacggcgt |
| 181 | cgaacgtgct gcaatggctt gctgctggct tctccatcct actgcttatg ttttacgcct |
| 241 | accaaacatg gaagtcaacc tgcggctggg aggagatcta tgtgtgcgct atcgagatgg |
| 301 | tcaaggtgat tctcgagttc ttcttcgagt ttaagaaccc gtccatgctg tatctagcca |
| 361 | caggccaccg cgtccagtgg ttgcgttacg ccgagtggct tctcacctgc ccggtcattc |
| 421 | tcattcacct gtcaaacctg acgggcttgt ccaacgacta cagcaggcgc accatgggtc |
| 481 | tgcttgtgtc tgatattggc acaattgtgt ggggcgccac ttccgccatg gccaccggat |
| 541 | acgtcaaggt catcttcttc tgcctgggtc tgtgttatgg tgctaacacg ttctttcacg |
| 601 | ctgccaaggc ctacatcgag ggttaccaca ccgtgccgaa gggccggtgt cgccaggtgg |
| 661 | tgactggcat ggcttggctc ttcttcgtat catggggtat gttccccatc ctgttcatcc |
| 721 | tcggccccga gggcttcggc gtcctgagcg tgtacggctc caccgtcggc cacaccatca |
| 781 | ttgacctgat gtcgaagaac tgctggggtc tgctcggcca ctacctgcgc gtgctgatcc |
| 841 | acgagcatat cctcatccac ggcgacattc gcaagaccac caaattgaac attggtggca |
| 901 | ctgagattga ggtcgagacg ctggtggagg acgaggccga ggctggcgcg gtcaacaagg |
| 961 | gcaccggcaa gtacgcctcc cgcgagtcct tcctggtcat gcgcgacaag atgaaggaga |
| 1021 | agggcattga cgtgcgcgcc tctctggaca acagcaagga ggtggagcag gagcaggccg |
| 1081 | ccagggctgc catgatgatg atgaacggca atggcatggg tatgggaatg ggaatgaacg |
| 1141 | gcatgaacgg aatgggcggt atgaacggga tggctggcgg cgccaagccc ggcctggagc |
| 1201 | tcactccgca gctacagccc ggccgcgtca tcctggcggt gccggacatc agcatggttg |
| 1261 | acttcttccg cgagcagttt gctcagctat cggtgacgta cgagctggtg ccggccctgg |
| 1321 | gcgctgacaa cacactggcg ctggttacgc aggcgcagaa cctgggcggc gtggactttg |
| 1381 | tgttgattca ccccgagttc ctgcgcgacc gctctagcac cagcatcctg agccgcctgc |
| 1441 | gcggcgcggg ccagcgtgtg gctgcgttcg gctgggcgca gctggggccc atgcgtgacc |
| 1501 | tgatcgagtc cgcaaacctg gacggctggc tggagggccc ctcgttcgga cagggcatcc |
| 1561 | tgccggccca catcgttgcc ctggtggcca agatgcagca gatgcgcaag atgcagcaga |
| 1621 | tgcagcagat tggcatgatg accggcggca tgaacggcat gggcggcggt atgggcggcg |
| 1681 | gcatgaacgg catgggcggc ggcaacggca tgaacaacat gggcaacggc atgggcggcg |
| 1741 | gcatgggcaa cggcatgggc ggcaatggca tgaacggaat gggtggcggc aacggcatga |
| 1801 | acaacatggg cggcaacgga atggccggca acggaatggg cggcggcatg ggcggcaacg |
| 1861 | gtatgggtgg ctccatgaac ggcatgagct ccggcgtggt ggccaacgtg acgccctccg |
| 1921 | ccgccggcgg catgggcggc atgatgaacg gcggcatggc tgcgccccag tcgcccggca |
| 1981 | tgaacggcgg ccgcctgggt accaacccgc tcttcaacgc cgcgccctca ccgctcagct |
| 2041 | cgcagctcgg tgccgaggca ggcatgggca gcatgggagg catgggcgga atgagcggaa |
| 2101 | tgggaggcat gggtggaatg gggggcatgg gcggcgccgg cgccgccacg acgcaggctg |
| 2161 | cgggcggcaa cgcggaggcg gagatgctgc agaatctcat gaacgagatc aatcgcctga |
| 2221 | agcgcgagct tggcgagtaa aaggctggag gccggtactg cgatacctgc gagctcgcgc |
| 2281 | gcctgactcg tcgtacacac ggctcaggag cacgcgcgcg tggacttctc aacctgtgtg |
| 2341 | caacgtatct agagcggcct gtgcgcgacc gtccgtgagc attccggtgc gatcttcccg |
| 2401 | ccttcgcacc gcaagttccc ttcctggccc tgctgcgcct gacgcatc |

A wild type (WT) Chop2 of the invention may be encoded by the following Chlamydomonas reinhardtii acop2 mRNA for archaeal-type opsin 2 amino acid sequence (GenBank Accession No. BAB68567, and SEQ ID NO: 8):

| | |
|---|---|
| 1 | mdyggalsav grellfvtnp vvvngsvlvp edqcycagwi esrgtngaqt asnvlqwlaa |
| 61 | gfsilllmfy ayqtwkstcg weeiyvcaie mvkvilefff efknpsmlyl atghrvqwlr |
| 121 | yaewlltcpv ilihlsnltg lsndysrrtm gllvsdigti vwgatsamat gyvkviffcl |
| 181 | glcygantff haakayiegy htvpkgrcrq vvtgmawlff vswgmfpilf ilgpegfgvl |
| 241 | svygstvght iidlmskncw gllghylrvl ihehilihgd irkttklnig gteievetlv |
| 301 | edeaeagavn kgtgkyasre sflvmrdkmk ekgidvrasl dnskeveqeq aaraammmmn |
| 361 | gngmgmgmgm ngmngmggmn gmaggakpgl eltpqlqpgr vilavpdism vdffreqfaq |
| 421 | lsvtyelvpa lgadntlalv tqaqnlggvd fvlihpeflr drsstsilsr lrgagqrvaa |
| 481 | fgwaqlgpmr dliesanldg wlegpsfgqg ilpahivalv akmqqmrkmq qmqqigmmtg |
| 541 | gmngmgggmg ggmngmgggn gmnnmgngmg ggmgngmggn gmngmgggng mnnmggngma |
| 601 | gngmgggmgg ngmggsmngm ssgvvanvtp saaggmggmm nggmaapqsp gmnggrlgtn |
| 661 | plfnaapspl ssqlgaeagm gsmggmggms gmggmggmgg mggagaattq aaggnaeaem |
| 721 | lqnlmneinr lkrelge |

### ChR2 Mutants

The present invention provides Chop2 mutants wherein one or more amino acids are mutated. In some embodiments, the Chop2 is the full-length polypeptide, such as SEQ ID NOs: 2, 4, 6, and 8, with at least one amino acid mutation. In some embodiments, the mutation is at amino acid 132 and/or amino acid 159. In some preferred embodiments, the amino acid at position 132 is mutated from a leucine to a cysteine or an alanine. In some preferred embodiments, the amino acid at position 159 is mutated from a threonine to an alanine, a cysteine, or a serine. In all embodiments, the Chop2 mutants form a functional ChR2 channel.

The present invention also encompasses Chop2 proteins and nucleic acids that encode a biologically active fragment or a conservative amino acid substitution or other mutation variant of Chop2. Non-limiting examples of useful fragments include polypeptides encoding amino acids 1-315 of the wild-type Chop2, i.e., SEQ ID NO: 26, wherein at least one amino acid is mutated or conservatively substituted, for example at amino acid positions 132 and/or 159. Smaller fragments of wild-type Chop2, wherein at least one amino acid is mutated or conservatively substituted (i.e., at amino acid positions 132 and/or 159) may also be useful in the present invention. Accordingly, Chop2 polypeptides and nucleic acids of the present invention further include, but are not limited to, biologically active fragments encoding amino acids 1-315, 1-310, 1-300, 1-275, 1-250, 1-225, 1-200, 1-175, or 1-160 of the wild-type Chop2, wherein at least one amino acid is mutated or conservatively substituted, for example at amino acid positions 132 and/or 159. In other embodiments, the Chop2 polypeptides and nucleic acids of the present invention can be up to, or about, 315 amino acids long, 310 amino acids long, 300 amino acids long, 275 amino acids long, 250 amino acids long, 225 amino acids long, 200 amino acids long, 175 amino acids long, or 160 amino acids long.

A single mutant Chop2 of the invention may be encoded by the following Synthetic construct hVChR1-mKate-betahChR2 (L132C) gene sequence (GenBank Accession No. JN836746, and SEQ ID NO: 9) with the following annotations, GFP sequence is in bold, L132C Chop2 sequence is underlined:

| | |
|---|---|
| 1 | atggattacc ctgtggcccg gtccctgatt gtaagatacc ccaccgatct gggcaatgga |
| 61 | accgtgtgca tgcccagagg acaatgctac tgcgaggggt ggctgaggag ccggggcact |
| 121 | agtatcgaaa aaaccatcgc tatcaccctc cagtgggtag tgttcgctct gtccgtagcc |
| 181 | tgtctcggct ggtatgcata ccaagcctgg agggctacct gtgggtggga ggaagtatac |
| 241 | gtggccctga tcgagatgat gaagtccatc atcgaggctt tccatgagtt cgactcccca |
| 301 | gccacactct ggctcagcag tgggaatggc gtagtgtgga tgagatatgg agagtggctg |
| 361 | ctgacctgtc ccgtcctgct cattcatctg tccaatctga ccgggctgaa agatgactac |
| 421 | tccaagagaa caatgggact gctggtgagt gacgtggggt gtattgtgtg gggagccacc |
| 481 | tccgccatgt gcactggatg gaccaagatc ctctttttcc tgatttccct ctcctatggg |
| 541 | atgtatacat acttccacgc cgctaaggtg tatattgagg ccttccacac tgtacctaaa |
| 601 | ggcatctgta gggagctcgt gcgggtgatg gcatggacct tctttgtggc ctgggggatg |
| 661 | ttccccgtgc tgttcctcct cggcactgag ggatttggcc acattagtcc ttacgggtcc |
| 721 | gcaattggac actccatcct ggatctgatt gccaagaata tgtggggggt gctgggaaat |
| 781 | tatctgcggg taaagatcca cgagcatatc ctgctgtatg gcgatatcag aaagaagcag |
| 841 | aaaatcacca ttgctggaca ggaaatggag gtggagacac tggtagcaga ggaggaggac |
| 901 | gggaccgcgg tcgccaccat ggtgtctaag ggcgaagagc tgattaagga gaacatgcac |
| 961 | atgaagctgt acatggaggg caccgtgaac aaccaccact tcaagtgcac atccgagggc |
| 1021 | gaaggcaagc cctacgaggg cacccagacc atgagaatca aggtggtcga gggcggccct |
| 1081 | ctccccttcg ccttcgacat cctggctacc agcttcatgt acggcagcaa aaccttcatc |
| 1141 | aaccacaccc agggcatccc cgacttcttt aagcagtcct tccctgaggg cttcacatgg |
| 1201 | gagagagtca ccacatacga agacgggggc gtgctgaccg ctacccagga caccagcctc |
| 1261 | caggacggct gcctcatcta caacgtcaag atcagagggg tgaacttccc atccaacggc |
| 1321 | cctgtgatgc agaagaaaac actcggctgg gaggcctcca ccgagatgct gtaccccgct |
| 1381 | gacggcggcc tggaaggcag agccgacatg gccctgaagc tcgtgggcgg gggccacctg |
| 1441 | atctgcaact tgaagaccac atacagatcc aagaaacccg ctaagaacct caagatgccc |
| 1501 | ggcgtctact atgtggacag aagactggaa agaatcaagg aggccgacaa agagacctac |
| 1561 | gtcgagcagc acgaggtggc tgtggccaga tactgcgacc tccctagcaa actggggcac |
| 1621 | aaacttaatt gcctgcagga gaagaagtca tgcagccagc gcatggccga attccggcaa |
| 1681 | tactgttgga acccggacac tgggcagatg ctgggccgca ccccagcccg gtgggtgtgg |
| 1741 | atcagcctgt actatgcagc tttctacgtg gtcatgactg ggctctttgc cttgtgcatc |
| 1801 | tatgtgctga tgcagaccat tgatccctac acccccgact accaggacca gttaaagtca |
| 1861 | ccgggggtaa ccttgagacc ggatgtgtat ggggaaagag ggctgcagat ttcctacaac |
| 1921 | atctctgaaa acagctctag acaggcccag atcaccggac gtccggagac tgagacattg |
| 1981 | ccaccggtgg actacggggg ggccctgagc gctgtgggca gagaactcct gttcgtgaca |
| 2041 | aatccagtcg tggtgaacgg ctccgtactc gtacccgagg atcagtgcta ttgcgcagga |
| 2101 | tggatcgaga gcagaggcac aaacggcgca cagactgcat ccaacgtgct ccagtggttg |
| 2161 | gccgcaggct tttccattct cctgctcatg ttttacgcct accagacttg gaagtccaca |
| 2221 | tgtggctggg aggaaatcta cgtgtgtgca atcgaaatgg tgaaggtgat cctggagttt |
| 2281 | ttcttcgaat ttaaaaaccc aagcatgctg tacctggcta ctggccacag agtgcagtgg |
| 2341 | ctgcggtatg ccgaatggct gctgacttgc ccagtgattt gcatccacct gtccaacctg |
| 2401 | actgggctgt ctaacgatta cagtaggaga acaatgggac tgctcgtatc cgacatcggc |
| 2461 | actatcgtat ggggcgcaac tagtgccatg gccactggat acgtgaaagt gatcttcttc |
| 2521 | tgcctgggac tctgctacgg agcaaacaca ttttttcatg ccgcaaaagc atatatcgag |
| 2581 | gggtatcata ccgtcccaaa gggccggtgt agacaagtgg tgactggcat ggcttggctg |
| 2641 | ttcttcgtgt cctgggggat gtttcccatc ctctttatcc tgggcccaga aggcttcggg |
| 2701 | gtgctgagtg tgtatggcag taccgtagga cacactatca ttgacctgat gagcaaaaac |
| 2761 | tgctgggggc tgctcggcca ctacctgaga gtactcatcc acgagcatat cctgattcat |
| 2821 | ggcgatatcc ggaaaactac caagctcaat atcgggggca ccgagattga agtggagaca |
| 2881 | ctcgtggagg acgaggccga ggccggagca gtgaacaaag gcactggcaa gtatgcctcc |
| 2941 | agagaatcct ttctggtgat gcgggacaaa atgaaggaga aaggcattga tgtacggtgc |
| 3001 | agtaatgcca aagccgtcga gactgatgtg tag |

A single mutant ChR2 of the invention may be encoded by the following Synthetic construct hVChR1-mKate-betahChR2 (L132C) amino acid sequence (GenBank Accession No. AER29839, and SEQ ID NO: 10) with the following annotations, GFP sequence is in bold, L132C Chop2 sequence is underlined:

| | |
|---|---|
| 1 | mdypvarsli vryptdlgng tvcmprgqcy cegwlrsrgt siektiaitl qwvvfalsva |
| 61 | clgwyayqaw ratcgweevy valiemmksi ieafhefdsp atlwlssgng vvwmrygewl |
| 121 | ltcpvllihl snltglkddy skrtmgllvs dvgcivwgat samctgwtki lfflislsyg |
| 181 | mytyfhaakv yieafhtvpk gicrelvrvm awtffvawgm fpvlfllgte gfghispygs |
| 241 | aighsildli aknmwgvlgn ylrvkihehi llygdirkkq kitiagqeme vetlvaeeed |
| 301 | gtavatmvsk geelikenmh mklymegtvn nhhfkctseg egkpyegtqt mrikvveggp |
| 361 | lpfafdilat sfmygsktfi nhtqgipdff kqsfpegftw ervttyedgg vltatqdtsl |
| 421 | qdgcliynvk irgvnfpsng pvmqkktlgw eastemlypa dgglegradm alklvggghl |
| 481 | icnlkttyrs kkpaknlkmp gvyyvdrrle rikeadkety veqhevavar ycdlpsklgh |
| 541 | klnclqekks csqrmaefrq ycwnpdtgqm lgrtparwvw islyyaafyv vmtglfalci |
| 601 | yvlmqtidpy tpdyqdqlks pgvtlrpdvy gerglqisyn isenssrqaq itgrpetetl |
| 661 | ppvdyggals avgrellfvt npvvvngsvl vpedqcycag wiesrgtnga qtasnvlqwl |
| 721 | aagfsilllm fyayqtwkst cgweeiyvca iemvkvilef ffefknpsml ylatghrvqw |
| 781 | lryaewlltc pvicihlsnl tglsndysrr tmgllvsdig tivwgatsam atgyvkviff |
| 841 | clglcygant ffhaakayie gyhtvpkgrc rqvvtgmawl ffvswgmfpi lfilgpegfg |
| 901 | vlsvygstvg htiidlmskn cwgllghylr vlihehilih gdirkttkln iggteievet |
| 961 | lvedeaeaga vnkgtgkyas resflvmrdk mkekgidvrc snakavetdv |

A single mutant Chop2 of the invention may be encoded by the following Synthetic construct hVChR1-mKate-betahChR2 (L132C) gene sequence (GenBank Accession No. JN836745, and SEQ ID NO: 11) with the following annotations, GFP sequence is in bold, L132C Chop2 sequence is underlined:

| | |
|---|---|
| 1 | atggattacc ctgtggcccg gtccctgatt gtaagatacc ccaccgatct gggcaatgga |
| 61 | accgtgtgca tgcccagagg acaatgctac tgcgaggggt ggctgaggag ccggggcact |
| 121 | agtatcgaaa aaaccatcgc tatcaccctc cagtgggtag tgttcgctct gtccgtagcc |
| 181 | tgtctcggct ggtatgcata ccaagcctgg agggctacct gtgggtggga ggaagtatac |
| 241 | gtggccctga tcgagatgat gaagtccatc atcgaggctt tccatgagtt cgactcccca |
| 301 | gccacactct ggctcagcag tgggaatggc gtagtgtgga tgagatatgg agagtggctg |
| 361 | ctgacctgtc ccgtcctgct cattcatctg tccaatctga ccgggctgaa agatgactac |
| 421 | tccaagagaa caatgggact gctggtgagt gacgtggggt gtattgtgtg gggagccacc |
| 481 | tccgccatgt gcactggatg gaccaagatc ctctttttcc tgatttccct ctcctatggg |
| 541 | atgtatacat acttccacgc cgctaaggtg tatattgagg ccttccacac tgtacctaaa |
| 601 | ggcatctgta gggagctcgt gcgggtgatg gcatggacct tctttgtggc ctgggggatg |
| 661 | ttccccgtgc tgttcctcct cggcactgag ggatttggcc acattagtcc ttacgggtcc |
| 721 | gcaattggac actccatcct ggatctgatt gccaagaata tgtggggggt gctgggaaat |
| 781 | tatctgcggg taaagatcca cgagcatatc ctgctgtatg gcgatatcag aaagaagcag |
| 841 | aaaatcacca ttgctggaca ggaaatggag gtggagacac tggtagcaga ggaggaggac |
| 901 | gggaccgcgg tcgccaccat ggtgtctaag ggcgaagagc tgattaagga gaacatgcac |
| 961 | atgaagctgt acatggaggg caccgtgaac aaccaccact tcaagtgcac atccgagggc |
| 1021 | gaaggcaagc cctacgaggg cacccagacc atgagaatca aggtggtcga gggcggccct |
| 1081 | ctccccttcg ccttcgacat cctggctacc agcttcatgt acggcagcaa aaccttcatc |
| 1141 | aaccacaccc agggcatccc cgacttcttt aagcagtcct tccctgaggg cttcacatgg |
| 1201 | gagagagtca ccacatacga agacgggggc gtgctgaccg ctacccagga caccagcctc |
| 1261 | caggacggct gcctcatcta caacgtcaag atcagagggg tgaacttccc atccaacggc |
| 1321 | cctgtgatgc agaagaaaac actcggctgg gaggcctcca ccgagatgct gtaccccgct |
| 1381 | gacggcggcc tggaaggcag agccgacatg gccctgaagc tcgtgggcgg gggccacctg |
| 1441 | atctgcaact tgaagaccac atacagatcc aagaaacccg ctaagaacct caagatgccc |
| 1501 | ggcgtctact atgtggacag aagactggaa agaatcaagg aggccgacaa agagacctac |
| 1561 | gtcgagcagc acgaggtggc tgtggccaga tactgcgacc tccctagcaa actggggcac |
| 1621 | aaacttaatt gcctgcagga gaagaagtca tgcagccagc gcatggccga attccggcaa |
| 1681 | tactgttgga acccggacac tgggcagatg ctgggccgca ccccagcccg gtgggtgtgg |
| 1741 | atcagcctgt actatgcagc tttctacgtg gtcatgactg ggctctttgc cttgtgcatc |
| 1801 | tatgtgctga tgcagaccat tgatccctac acccccgact accaggacca gttaaagtca |
| 1861 | ccgggggtaa ccttgagacc ggatgtgtat ggggaaagag ggctgcagat ttcctacaac |
| 1921 | atctctgaaa acagctctag acaggcccag atcaccggac gtccggagac tgagacattg |
| 1981 | ccaccggtgg actacggggg ggccctgagc gctgtgggca gagaactcct gttcgtgaca |
| 2041 | aatccagtcg tggtgaacgg ctccgtactc gtacccgagg atcagtgcta ttgcgcagga |
| 2101 | tggatcgaga gcagaggcac aaacggcgca cagactgcat ccaacgtgct ccagtggttg |
| 2161 | gccgcaggct tttccattct cctgctcatg ttttacgcct accagacttg gaagtccaca |
| 2221 | tgtggctggg aggaaatcta cgtgtgtgca atcgaaatgg tgaaggtgat cctggagttt |
| 2281 | ttcttcgaat ttaaaaaccc aagcatgctg tacctggcta ctggccacag agtgcagtgg |
| 2341 | ctgcggtatg ccgaatggct gctgacttgc ccagtgattc tgatccacct gtccaacctg |
| 2401 | actgggctgt ctaacgatta cagtaggaga acaatgggac tgctcgtatc cgacatcggc |
| 2461 | actatcgtat ggggcgcaac tagtgccatg gccactggat acgtgaaagt gatcttcttc |
| 2521 | tgcctgggac tctgctacgg agcaaacaca ttttttcatg ccgcaaaagc atatatcgag |
| 2581 | gggtatcata ccgtcccaaa gggccggtgt agacaagtgg tgactggcat ggcttggctg |
| 2641 | ttcttcgtgt cctgggggat gtttcccatc ctctttatcc tgggcccaga aggcttcggg |
| 2701 | gtgctgagtg tgtatggcag taccgtagga cacactatca ttgacctgat gagcaaaaac |
| 2761 | tgctgggggc tgctcggcca ctacctgaga gtactcatcc acgagcatat cctgattcat |
| 2821 | ggcgatatcc ggaaaactac caagctcaat atcgggggca ccgagattga agtggagaca |
| 2881 | ctcgtggagg acgaggccga ggccggagca gtgaacaaag gcactggcaa gtatgcctcc |
| 2941 | agagaatcct ttctggtgat gcgggacaaa atgaaggaga aaggcattga tgtacggtgc |
| 3001 | agtaatgcca aagccgtcga gactgatgtg tag |

A single mutant Chop2 of the invention may be encoded by the following Synthetic construct hVChR1-mKate-betahChR2 (L132C) amino acid sequence (GenBank Accession No. AER29838, and SEQ ID NO: 12) with the following annotations, GFP sequence is in bold, L132C Chop2 sequence is undefined:

| | |
|---|---|
| 1 | mdypvarsli vryptdlgng tvcmprgqcy cegwlrsrgt siektiaitl qwvvfalsva |
| 61 | clgwyayqaw ratcgweevy valiemmksi ieafhefdsp atlwlssgng vvwmrygewl |
| 121 | ltcpvllihl snltglkddy skrtmgllvs dvgcivwgat samctgwtki lfflislsyg |
| 181 | mytyfhaakv yieafhtvpk gicrelvrvm awtffvawgm fpvlfllgte gfghispygs |
| 241 | aighsildli aknmwgvlgn ylrvkihehi llygdirkkq kitiagqeme vetlvaeeed |
| 301 | gtavatmvsk geelikenmh mklymegtvn nhhfkctseg egkpyegtqt mrikvveggp |
| 361 | lpfafdilat sfmygsktfi nhtqgipdff kqsfpegftw ervttyedgg vltatqdtsl |
| 421 | qdgcliynvk irgvnfpsng pvmqkktlgw eastemlypa dgglegradm alklvggghl |
| 481 | icnlkttyrs kkpaknlkmp gvyyvdrrle rikeadkety veqhevavar ycdlpsklgh |
| 541 | klnclqekks csqrmaefrq ycwnpdtgqm lgrtparwvw islyyaafyv vmtglfalci |
| 601 | yvlmqtidpy tpdyqdqlks pgvtlrpdvy gerglqisyn isenssrqaq itgrpetetl |
| 661 | ppvdyggals avgrellfvt npvvvngsvl vpedqcycag wiesrgtnga qtasnvlqwl |
| 721 | aagfsilllm fyayqtwkst cgweeiyvca iemvkvilef ffefknpsml ylatghrvqw |
| 781 | lryaewlltc pvilihlsnl tglsndysrr tmgllvsdig tivwgatsam atgyvkviff |
| 841 | clglcygant ffhaakayie gyhtvpkgrc rqvvtgmawl ffvswgmfpi lfilgpegfg |
| 901 | vlsvygstvg htiidlmskn cwgllghylr vlihehilih gdirkttkln iggteievet |
| 961 | lvedeaeaga vnkgtgkyas resflvmrdk mkekgidvrc snakavetdv |

A L132C single mutant Chop2 of the invention may be encoded by the following amino acid sequence (positions 132 underlined and bolded, SEQ ID NO: 13):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ICIHLSNLTG LSNDYSRRTM GLLVSDIGTI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A T159C single mutant Chop2 of the invention may be encoded by the following amino acid sequence (positions 159 underlined and bolded, SEQ ID NO: 14):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ILIHLSNLTG LSNDYSRRTM GLLVSDIGCI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A L132C/T159C double mutant Chop2 of the invention may be encoded by the following nucleotide sequence (SEQ ID NO: 15):

| | |
|---|---|
| 1 | atggactacg ggggggctct gtctgctgtc gggagggaac tgctgtttgt gactaaccct |
| 61 | gtcgtcgtga acgggagtgt gctggtccct gaggaccagt gctactgtgc cggctggatc |
| 121 | gaatcacgcg gaaccaacgg ggcccagaca gctagcaatg tgctgcagtg gctggccgct |
| 181 | gggtttagta tcctgctgct gatgttctac gcctatcaga cttggaagtc aacctgcggc |
| 241 | tgggaggaaa tctacgtgtg cgctattgag atggtgaaag tgatcctgga gttcttcttc |
| 301 | gagttcaaga acccaagcat gctgtacctg gctactggac accgagtgca gtggctgaga |
| 361 | tatgcagaat ggctgctgac atgccccgtc atctgcattc acctgtccaa cctgacaggc |
| 421 | ctgagcaatg actactccag gagaactatg ggactgctgg tgtccgacat cggctgcatt |
| 481 | gtctggggag caacttctgc tatggcaacc ggatacgtga aggtcatctt tttctgcctg |
| 541 | gggctgtgct atggcgcaaa tacctttttc cacgcagcca aggcctacat tgaggggtat |
| 601 | cataccgtgc caaaaggccg gtgccgacag gtggtcacag gaatggcttg gctgtttttc |
| 661 | gtctcttggg gaatgtttcc catcctgttc attctggggc ctgaagggtt cggcgtgctg |
| 721 | tctgtctacg gaagtacagt ggggcatact atcattgacc tgatgtccaa aaactgttgg |
| 781 | ggcctgctgg gacactatct gagagtgctg atccacgagc atatcctgat tcatggcgat |
| 841 | attcggaaga ccacaaaact gaatatcggc ggaaccgaga ttgaagtgga aacactggtg |
| 901 | gaagacgagg ctgaggctgg ggctgtgaac aaggggactg gcaaa |

A L132C/T159C double mutant Chop2 of the invention may be encoded by the following amino acid sequence (positions 132 and 159 underlined and bolded, SEQ ID NO: 16):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ICIHLSNLTG LSNDYSRRTM GLLVSDIGCI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A T159S single mutant Chop2 of the invention may be encoded by the following amino acid sequence (positions 159 underlined and bolded, SEQ ID NO: 17):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ILIHLSNLTG LSNDYSRRTM GLLVSDIGSI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A L132C/T159S double mutant Chop2 of the invention may be encoded by the following nucleotide sequence (SEQ ID NO: 18):

| | |
|---|---|
| 1 | atggactacg ggggggctct gtctgctgtc gggagggaac tgctgtttgt gactaaccct |
| 61 | gtcgtcgtga acgggagtgt gctggtccct gaggaccagt gctactgtgc cggctggatc |
| 121 | gaatcacgcg gaaccaacgg ggcccagaca gctagcaatg tgctgcagtg gctggccgct |
| 181 | gggtttagta tcctgctgct gatgttctac gcctatcaga cttggaagtc aacctgcggc |
| 241 | tgggaggaaa tctacgtgtg cgctattgag atggtgaaag tgatcctgga gttcttcttc |
| 301 | gagttcaaga acccaagcat gctgtacctg gctactggac accgagtgca gtggctgaga |
| 361 | tatgcagaat ggctgctgac atgccccgtc atctgcattc acctgtccaa cctgacaggc |
| 421 | ctgagcaatg actactccag gagaactatg ggactgctgg tgtccgacat cggcagcatt |
| 481 | gtctggggag caacttctgc tatggcaacc ggatacgtga aggtcatctt tttctgcctg |
| 541 | gggctgtgct atggcgcaaa tacctttttc cacgcagcca aggcctacat tgaggggtat |
| 601 | cataccgtgc caaaaggccg gtgccgacag gtggtcacag gaatggcttg gctgtttttc |
| 661 | gtctcttggg gaatgtttcc catcctgttc attctggggc ctgaagggtt cggcgtgctg |
| 721 | tctgtctacg gaagtacagt ggggcatact atcattgacc tgatgtccaa aaactgttgg |
| 781 | ggcctgctgg gacactatct gagagtgctg atccacgagc atatcctgat tcatggcgat |
| 841 | attcggaaga ccacaaaact gaatatcggc ggaaccgaga ttgaagtgga aacactggtg |
| 901 | gaagacgagg ctgaggctgg ggctgtgaac aaggggactg gcaaa |

A L132C/T159S double mutant Chop2 of the invention may be encoded by the following amino acid sequence (positions 132 and 159 underlined and bolded, SEQ ID NO: 19):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ICIHLSNLTG LSNDYSRRTM GLLVSDIGSI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A L132A single mutant Chop2 of the invention may be encoded by the following amino acid sequence (position 132 underlined and bolded, SEQ ID NO: 20):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV IAIHLSNLTG LSNDYSRRTM GLLVSDIGTI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A L132A/T159C double mutant Chop2 of the invention may be encoded by the following nucleotide sequence (SEQ ID NO: 21):

| | |
|---|---|
| 1 | ATGGACTACG GGGGGGCTCT GTCTGCTGTC GGGAGGGAAC TGCTGTTTGT GACTAACCCT |
| 61 | GTCGTCGTGA ACGGGAGTGT GCTGGTCCCT GAGGACCAGT GCTACTGTGC CGGCTGGATC |
| 121 | GAATCACGCG GAACCAACGG GGCCCAGACA GCTAGCAATG TGCTGCAGTG GCTGGCCGCT |
| 181 | GGGTTTAGTA TCCTGCTGCT GATGTTCTAC GCCTATCAGA CTTGGAAGTC AACCTGCGGC |
| 241 | TGGGAGGAAA TCTACGTGTG CGCTATTGAG ATGGTGAAAG TGATCCTGGA GTTCTTCTTC |
| 301 | GAGTTCAAGA ACCCAAGCAT GCTGTACCTG GCTACTGGAC ACCGAGTGCA GTGGCTGAGA |
| 361 | TATGCAGAAT GGCTGCTGAC ATGCCCCGTC ATCGCCATTC ACCTGTCCAA CCTGACAGGC |
| 421 | CTGAGCAATG ACTACTCCAG GAGAACTATG GGACTGCTGG TGTCCGACAT CGGCTGCATT |
| 481 | GTCTGGGGAG CAACTTCTGC TATGGCAACC GGATACGTGA AGGTCATCTT TTTCTGCCTG |
| 541 | GGGCTGTGCT ATGGCGCAAA TACCTTTTTC CACGCAGCCA AGGCCTACAT TGAGGGGTAT |
| 601 | CATACCGTGC CAAAAGGCCG GTGCCGACAG GTGGTCACAG GAATGGCTTG GCTGTTTTTC |
| 661 | GTCTCTTGGG GAATGTTTCC CATCCTGTTC ATTCTGGGGC CTGAAGGGTT CGGCGTGCTG |
| 721 | TCTGTCTACG GAAGTACAGT GGGGCATACT ATCATTGACC TGATGTCCAA AAACTGTTGG |
| 781 | GGCCTGCTGG GACACTATCT GAGAGTGCTG ATCCACGAGC ATATCCTGAT TCATGGCGAT |
| 841 | ATTCGGAAGA CCACAAAACT GAATATCGGC GGAACCGAGA TTGAAGTGGA AACACTGGTG |
| 901 | GAAGACGAGG CTGAGGCTGG GGCTGTGAAC AAGGGGACTG GCAAA |

A L132A/T159C double mutant Chop2 of the invention may be encoded by the following amino acid sequence (positions 132 and 159 underlined and bolded, SEQ ID NO: 22):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV IAIHLSNLTG LSNDYSRRTM GLLVSDIGCI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A T159A single mutant Chop2 of the invention may be encoded by the following amino acid sequence (position 159 underlined and bolded, SEQ ID NO: 23):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ILIHLSNLTG LSNDYSRRTM GLLVSDIGAI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A L132C/T159A double mutant Chop2 of the invention may be encoded by the following nucleotide sequence (SEQ ID NO: 24):

| | |
|---|---|
| 1 | atggactacg ggggggctct gtctgctgtc gggagggaac tgctgtttgt gactaaccct |
| 61 | gtcgtcgtga acgggagtgt gctggtccct gaggaccagt gctactgtgc cggctggatc |
| 121 | gaatcacgcg gaaccaacgg ggcccagaca gctagcaatg tgctgcagtg gctggccgct |
| 181 | gggtttagta tcctgctgct gatgttctac gcctatcaga cttggaagtc aacctgcggc |
| 241 | tgggaggaaa tctacgtgtg cgctattgag atggtgaaag tgatcctgga gttcttcttc |
| 301 | gagttcaaga acccaagcat gctgtacctg gctactggac accgagtgca gtggctgaga |
| 361 | tatgcagaat ggctgctgac atgccccgtc atctgcattc acctgtccaa cctgacaggc |
| 421 | ctgagcaatg actactccag gagaactatg ggactgctgg tgtccgacat cggcgccatt |
| 481 | gtctggggag caacttctgc tatggcaacc ggatacgtga aggtcatctt tttctgcctg |
| 541 | gggctgtgct atggcgcaaa tacctttttc cacgcagcca aggcctacat tgaggggtat |
| 601 | cataccgtgc caaaaggccg gtgccgacag gtggtcacag gaatggcttg gctgtttttc |
| 661 | gtctcttggg gaatgtttcc catcctgttc attctggggc ctgaagggtt cggcgtgctg |
| 721 | tctgtctacg gaagtacagt ggggcatact atcattgacc tgatgtccaa aaactgttgg |
| 781 | ggcctgctgg gacactatct gagagtgctg atccacgagc atatcctgat tcatggcgat |
| 841 | attcggaaga ccacaaaact gaatatcggc ggaaccgaga ttgaagtgga aacactggtg |
| 901 | gaagacgagg ctgaggctgg ggctgtgaac aaggggactg gcaaa |

A L132C/T159A double mutant Chop2 of the invention may be encoded by the following amino acid sequence (positions 132 and 159 underlined and bolded, SEQ ID NO: 25):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ICIHLSNLTG LSNDYSRRTM GLLVSDIGAI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

A wild type (WT) Chop2 of the invention may be encoded by the following amino acid sequence (SEQ ID NO: 26):

| | |
|---|---|
| 1 | MDYGGALSAV GRELLFVTNP VVVNGSVLVP EDQCYCAGWI ESRGTNGAQT ASNVLQWLAA |
| 61 | GFSILLLMFY AYQTWKSTCG WEEIYVCAIE MVKVILEFFF EFKNPSMLYL ATGHRVQWLR |
| 121 | YAEWLLTCPV ILIHLSNLTG LSNDYSRRTM GLLVSDIGTI VWGATSAMAT GYVKVIFFCL |
| 181 | GLCYGANTFF HAAKAYIEGY HTVPKGRCRQ VVTGMAWLFF VSWGMFPILF ILGPEGFGVL |
| 241 | SVYGSTVGHT IIDLMSKNCW GLLGHYLRVL IHEHILIHGD IRKTTKLNIG GTEIEVETLV |
| 301 | EDEAEAGAVN KGTGK |

Mutant ChR2 proteins of the invention also demonstrate slower channel kinetics. Higher light sensitivity was found to correlate with slower channel kinetics, indicating a trade-off between light sensitivity and channel kinetics. Chop2 proteins that form the ChR2 proteins of the present invention may also comprise additional mutations or modifications that may improve channel kinetics, or increase the deactivation rate, of the ChR2. Particularly preferred ChR2 mutants balance the threshold of light sensitivity with channel kinetics.

### Compositions and Kits

Compositions and kits of the invention comprise at least one nucleic acid molecule or polypeptide molecule that encodes a mutant Chop2 protein, and the resulting ChR2, of the invention. The at least one nucleic acid molecule or polypeptide molecule that encodes a mutant Chop2 protein of the invention may further include a pharmaceutically-acceptable carrier. Kits of the invention further include instructions for administering a composition of the invention to a subject.

### Therapeutic Uses

Mutations were made on a codon optimized Chop2-GFP fusion protein to create single and double mutations at the L132 (Leucine 132) and T159 (threonine 159) sites. The functional properties of each mutant ChR2, or a combination thereof, were first examined in HEK cells. AAV2 virus vectors carrying mutant Chop2-GFP constructs driven by CAG promoter were made and injected intravitreally into the eyes of adult mice. Mutant Chop2-mediated light responses were examined by using multi-electrode array recordings from whole-mount retinas.

Single mutant ChR2, i.e., L132 and T159C, markedly lower the threshold light intensity that is required to evoke a ChR2-mediated photocurrent. Moreover, several double mutant ChR2 variants, including L132C/T159C, L132A/T159C, and L132C/T159S, were found to further increase the photocurrent above the results of any single mutant ChR2 at low light intensities. The double mutants exhibited a slower off-rate, which is likely to contribute to the increased photocurrent at the low light intensities. Spiking activity of retinal ganglion cells mediated by the L132C/T159C double mutant was observed at the light intensity of 10¹³ photon/cm²/s and at the wavelength of 473 nm. This light level is about 1.5 to 2 log units lower than the light level that is required to elicit the spiking activity with wild-type ChR2. The spike firing of retinal ganglion cells expressing L132C/T159C could follow a light flicker frequency of up to 15 Hz. Ongoing studies are evaluating the long-term expression and safety of mutant ChR2s of the invention in retinal neurons.

Furthermore, expression of the mutant Chop2 proteins, and the resulting ChR2 proteins, of the present invention was not found to cause neurotoxicity of up to two months after viral injection in mice, demonstrating the safety of the present invention for therapeutic use.

Vectors for use in the present invention can include various viral vectors, such as plasmids and recombinant viruses, i.e., recombinant adeno-associated virus (rAAV), recombinant adenoviruses, recombinant retroviruses, recombinant lentiviruses, and other viruses known in the art.

In some embodiments, the expression of the Chop2 proteins of the present invention is driven by a constitutive promoter, i.e., CAG promoter, CMV promoter, LTR. In other embodiments, the promoter is an inducible or a cell-specific promoter. Cell type-specific promoters that enable Chop2 protein expression in specific subpopulations of cells, i.e., retinal neuron cells or degenerating cells, may be preferred. These cells may include, but are not limited to, a retinal ganglion cell, a photoreceptor cell, a bipolar cell, a rod bipolar cell, an ON-type cone bipolar cell, a retinal ganglion cell, a photosensitive retinal ganglion cell, a horizontal cell, an amacrine cell, or an AII amacrine cell. Cell type-specific promoters are well known in the art. Particularly preferred cell type-specific promoters include, but are not limited to mGluR6, NK-3, and Pcp2 (L7).

In some embodiments, use of different opsin genes in addition to the mutant Chop2 proteins of the present invention and targeted gene expression may further increase light sensitivity or improve vision. Visual information is processed through the retina through two pathways: an ON pathway which signals the light ON, and an OFF pathway which signals the light OFF. The existence of the ON and OFF pathway is important for the enhancement of contrast sensitivity. The visual signal in the ON pathway is relay from ON-cone bipolar cells to ON ganglion cells. Both ON-cone bipolar cells and ON-ganglion cells are depolarized in response to light. On the other hand, the visual signal in the OFF pathway is carried from OFF-cone bipolar cells to OFF ganglion cells. Both OFF-cone bipolar cells and OFF-ganglion cells are hypopolarized in response to light. Rod bipolar cells, which are responsible for the ability to see in dim light (scotopic vision), are ON bipolar cells (depolarized in response to light). Rod bipolar cells relay the vision signal through AII amacrine cells (an ON type retinal cells) to ON an OFF cone bipolar cells.

Accordingly, a dual rhodopsin system can be used to recapitulate the ON and OFF pathways integral to visual processing and acuity. Briefly, a Chop2 protein of the present invention can be specifically targeted to ON type retinal neurons (i.e., ON type ganglion cells and/or ON type bipolar cells), while a hypopolarizing light sensor (i.e., halorhodopsin or other chloride pump known in the art) can be targeted to OFF type retinal neurons (i.e. OFF type ganglion cells and/or OFF type bipolar cells) to create ON and OFF pathways. The specific targeting to preferred cell subpopulations can be achieved through the use of different cell type-specific promoters. For example, Chop2 expression may be driven by the mGluR6 promoter for targeted expression in ON-type retinal neurons (i.e., ON type ganglion cells and/or ON type bipolar cells) while a hypopolarizing channel, such as halorhodopsin, expression is driven by the NK-3 promoter for targeted expression in OFF-type retinal neurons (i.e., OFF type ganglion cells and/or OFF type bipolar cells).

An alternative approach to restore ON and OFF pathways in the retina is achieved by, expressing a depolarizing light sensor, such as ChR2, to rod bipolar cells or AII amacrine. In this approach, the depolarization of rod bipolar cells or AII amacrine cells can lead to the ON and OFF responses at the levels of cone bipolar cells and the downstream retinal ganglion cells. Thus, the ON and OFF pathways that are inherent in the retina are maintained.

The present invention can be formulated to a pharmaceutical composition or medicament suitable for administration into a subject or patient. Suitable routes of administration include, for example, intravitreal, intraocular, or subretinal injection.

Such formulations comprise a pharmaceutically and/or physiologically acceptable vehicle, diluent, carrier or excipient, such as buffered saline or other buffers, e.g., HEPES, to maintain physiologic pH. For a discussion of such components and their formulation, see, generally, Gennaro, A E., Remington: The Science and Practice of Pharmacy. Lippincott Williams & Wilkins Publishers: 2003 or latest edition). See also, WO00/15822. If the preparation is to be stored for long periods, it may be frozen, for example, in the presence of glycerol.

The pharmaceutical composition described above is administered to a subject having a visual or blinding disease by any appropriate route, preferably by intravitreal or subretinal injection, depending on the retinal layer being targeted.

Disclosures from Bennett and colleagues (cited herein) concern targeting of retinal pigment epithelium—the most distal layer from the vitreal space. According to the present invention, the Chop2 construct or polypeptide is targeted to retinal cells, i.e., retinal ganglion cells or bipolar cells. Such cells are known to be reasonably well-accessible to intravitreal injection as disclosed herein. Intravitreal and/or subretinal injection can provide the necessary access to the bipolar cells, especially in circumstances in which the photoreceptor cell layer is absent due to degeneration—which is the case in certain forms of degeneration that the present invention is intended to overcome.

To test for the vector's ability to express the Chop2 mutants of the present invention, specifically in mammalian retinal neurons, by AAV-mediated delivery, a combination of a preferred promoter sequence linked to a reporter gene such as LacZ or GFP linked to a SV40 poly A sequence can be inserted into a plasmid and packaged into rAAV virus particles, concentrated, tested for contaminating adenovirus and titered for rAAV using an infectious center assay. The right eyes of a number of test subjects, preferably inbred mice, can be injected sub-retinally with about 1 μl of the rAAV preparation (e.g., greater than about 10¹⁰ infectious units ml). Two weeks later, the right (test) and left (control) eyes of half the animals may be removed, fixed and stained with an appropriate substrate or antibody or other substance to reveal the presence of the reporter gene. A majority of the test retinas in injected eyes will exhibited a focal stained region, e.g., blue for LacZ/Xgal, or green for GFP consistent with a subretinal bleb of the injected virus creating a localized retinal detachment. All control eyes may be negative for the reporter gene product. Reporter gene expression examined in mice sacrificed at later periods is detected for at least 10 weeks post-injection, which suggests persistent expression of the reporter transgene.

In one embodiment, the Chop2 constructs are packaged in adenoviral vectors for transgene delivery. An effective amount of rAAV virions carrying a nucleic acid sequence encoding the Chop2 DNA under the control of the promoter of choice, preferably a constitutive CMV promoter or a cell-specific promoter such as mGluR6, is preferably in the range of between about 10¹⁰ to about 10¹³ rAAV infectious units in a volume of between about 150 and about 800 μl per injection. The rAAV infectious units can be measured according to McLaughlin, S K et al., 1988, J Virol 62:1963. More preferably, the effective amount is between about 10¹⁰ and about 10¹² rAAV infectious units and the injection volume is preferably between about 250 and about 500 μl. Other dosages and volumes, preferably within these ranges but possibly outside them, may be selected by the treating professional, taking into account the physical state of the subject (preferably a human), who is being treated, including, age, weight, general health, and the nature and severity of the particular ocular disorder.

It may also be desirable to administer additional doses (“boosters”) of the present nucleic acid(s) or rAAV compositions. For example, depending upon the duration of the transgene expression within the ocular target cell, a second treatment may be administered after 6 months or yearly, and may be similarly repeated. Neutralizing antibodies to AAV are not expected to be generated in view of the routes and doses used, thereby permitting repeat treatment rounds.

The need for such additional doses can be monitored by the treating professional using, for example, well-known electrophysiological and other retinal and visual function tests and visual behavior tests. The treating professional will be able to select the appropriate tests applying routine skill in the art. It may be desirable to inject larger volumes of the composition in either single or multiple doses to further improve the relevant outcome parameters.

### Ocular Disorders

The ocular disorders for which the present Chop2 proteins, and the resulting ChR2 proteins, are intended and may be used to improve one or more parameters of vision include, but are not limited to, developmental abnormalities that affect both anterior and posterior segments of the eye. Anterior segment disorders include glaucoma, cataracts, corneal dystrophy, keratoconus. Posterior segment disorders include blinding disorders caused by photoreceptor malfunction and/or death caused by retinal dystrophies and degenerations. Retinal disorders include congenital stationary night blindness, age-related macular degeneration, congenital cone dystrophies, and a large group of retinitis-pigmentosa (RP)-related disorders. These disorders include genetically pre-disposed death of photoreceptor cells, rods and cones in the retina, occurring at various ages. Among those are severe retinopathies, such as subtypes of RP itself that progresses with age and causes blindness in childhood and early adulthood and RP-associated diseases, such as genetic subtypes of LCA, which frequently results in loss of vision during childhood, as early as the first year of life. The latter disorders are generally characterized by severe reduction, and often complete loss of photoreceptor cells, rods and cones. (Trabulsi, E I, ed., Genetic Diseases of the Eye. Oxford University Press, NY, 1998).

In particular, the Chop2 and ChR2 proteins of the present invention useful for the treatment and/or restoration of at least partial vision to subjects that have lost vision due to ocular disorders, such as RPE-associated retinopathies, which are characterized by a long-term preservation of ocular tissue structure despite loss of function and by the association between function loss and the defect or absence of a normal gene in the ocular cells of the subject. A variety of such ocular disorders are known, such as childhood onset blinding diseases, retinitis pigmentosa, macular degeneration, and diabetic retinopathy, as well as ocular blinding diseases known in the art. It is anticipated that these other disorders, as well as blinding disorders of presently unknown causation which later are characterized by the same description as above, may also be successfully treated by the Chop2 and ChR2 proteins of the present invention. Thus, the particular ocular disorder treated by the present invention may include the above-mentioned disorders and a number of diseases which have yet to be so characterized.

### Optogenetics

The emerging field of optogenetics involves the combination of genetic and optical methods to control specific events in targeted cells of a living tissue. Optogenetics may be used within freely moving mammals and other animals. Moreover, the temporal precision (millisecond-timescale) of optogenetic methods are sufficient to function within intact biological systems.

The instant invention provides Chop2-gene therapy to retinal tissues of the eye, by introducing into retinal cells a nucleic acid or polypeptide encoding for at least one mutant form of Chop2. Mutant Chop2/ChR2 proteins of the invention are specifically adapted to be light-activated at lower thresholds of light intensities than their wild type counterparts. Accordingly, the mutant Chop2/ChR2 proteins of the invention can be used to activate cells of the retina and visual system using less damaging sources of illumination. The mutant Chop2/ChR2 proteins also conduct larger photocurrents upon activation, resulting in a more robust or efficacious response from the mutant Chop2/ChR2-expressing cells.

For example, mutant Chop2 proteins of the invention are administered to a subject through local, intravitreous or subretinal, injection of a nucleic acid molecule encoding a mutant Chop2, a mutant Chop2 polypeptide molecule, or a cell expressing a mutant Chop2/ChR2. Retinal cells of the subject express the mutant Chop2 proteins within the plasma membrane. When the transfected or transformed retinal cells encounter light radiation, the transfected or transformed retinal cells transduce an improved or restored signal.

These methods may be used in subjects of normal and/or impaired vision. Chop2/ChR2 mutants of the invention may preserve, improve, or restore vision. Moreover, Chop2/ChR2 mutants of the invention are used to preserve, improve, or restore the transduction of non-visual information from photosensitive retinal ganglion cells to the brain.

The term “vision” as used herein is defined as the ability of an organism to usefully detect light as a stimulus for differentiation or action. Vision is intended to encompass the following:
1. Light detection or perception—the ability to discern whether or not light is present;
2. Light projection—the ability to discern the direction from which a light stimulus is coming;
3. Resolution—the ability to detect differing brightness levels (i.e., contrast) in a grating or letter target; and
4. Recognition—the ability to recognize the shape of a visual target by reference to the differing contrast levels within the target.

Thus, “vision” includes the ability to simply detect the presence of light. The polypeptides and polynucleotides encoding mutant Chop2 of the present invention can be used to improve or restore vision, wherein the improvement or restoration in vision includes, for example, increases in light detection or perception, increase in light sensitivity or photosensitivity in response to a light stimulus, increase in the ability to discern the direction from which a light stimulus is coming, increase in the ability to detect differing brightness levels, increase in the ability to recognize the shape of a visual target, and increases in visual evoked potential or transmission from the retina to the cortex. As such, improvement or restoration of vision may or may not include full restoration of sight, i.e., wherein the vision of the patient treated with the present invention is restored to the degree to the vision of a non-affected individual. The visual recovery described in the animal studies described below may, in human terms, place the person on the low end of vision function by increasing one aspect of vision (i.e., light sensitivity, or visual evoked potential) without restoring full sight. Nevertheless, placement at such a level would be a significant benefit because these individuals could be trained in mobility and potentially in low order resolution tasks which would provide them with a greatly improved level of visual independence compared to total blindness. Even basic light perception can be used by visually impaired individuals, whose vision is improved using the present compositions and methods, to accomplish specific daily tasks and improve general mobility, capability, and quality of life.

The degree of restoration of vision can be determined through the measurement of vision before, and preferably after, administering a vector comprising, for example, DNA encoding Chop2. Vision can be measured using any of a number of methods well-known in the art or methods not yet established. Vision, as improved or restored by the present invention, can be measured by any of the following visual responses:
1. a light detection response by the subject after exposure to a light stimulus—in which evidence is sought for a reliable response of an indication or movement in the general direction of the light by the subject individual when the light it is turned on;
2. a light projection response by the subject after exposure to a light stimulus in which evidence is sought for a reliable response of indication or movement in the specific direction of the light by the individual when the light is turned on;
3 light resolution by the subject of a light vs. dark patterned visual stimulus, which measures the subject's capability of resolving light vs dark patterned visual stimuli as evidenced by:
a. the presence of demonstrable reliable optokinetically produced nystagmoid eye movements and/or related head or body movements that demonstrate tracking of the target (see above) and/or
b. the presence of a reliable ability to discriminate a pattern visual stimulus and to indicate such discrimination by verbal or non-verbal means, including, for example pointing, or pressing a bar or a button: or
4. electrical recording of a visual cortex response to a light flash stimulus or a pattern visual stimulus, which is an endpoint of electrical transmission from a restored retina to the visual cortex, also referred to as the visual evoked potential (VEP). Measurement may be by electrical recording on the scalp surface at the region of the visual cortex, on the cortical surface, and/or recording within cells of the visual cortex.

Thus, improvement or restoration of vision, according to the present invention, can include, but is not limited to: increases in amplitude or kinetics of photocurrents or electrical response in response to light stimulus in the retinal cells, increases in light sensitivity (i.e., lowering the threshold light intensity required for initiating a photocurrent or electrical response in response to light stimulus, thereby requiring less or lower light to evoke a photocurrent) of the retinal cells, increases in number or amplitude of light-evoked spiking or spike firings, increases in light responses to the visual cortex, which includes increasing in visual evoked potential transmitted from the retina or retinal cells to the visual cortex or the brain.

Both in vitro and in vivo studies to assess the various parameters of the present invention may be used, including recognized animal models of blinding human ocular disorders. Large animal models of human retinopathy, e.g., childhood blindness, are useful. The examples provided herein allow one of skill in the art to readily anticipate that this method may be similarly used in treating a range of retinal diseases.

While earlier studies by others have demonstrated that retinal degeneration can be retarded by gene therapy techniques, the present invention demonstrates a definite physiological recovery of function, which is expected to generate or improve various parameters of vision, including behavioral parameters.

Behavioral measures can be obtained using known animal models and tests, for example performance in a water maze, wherein a subject in whom vision has been preserved or restored to varying extents will swim toward light (Hayes, J M et al., 1993, Behav Genet 23:395-403).

In models in which blindness is induced during adult life or congenital blindness develops slowly enough that the individual experiences vision before losing it, training of the subject in various tests may be done. In this way, when these tests are re-administered after visual loss to test the efficacy of the present compositions and methods for their vision-restorative effects, animals do not have to learn the tasks de novo while in a blind state. Other behavioral tests do not require learning and rely on the instinctiveness of certain behaviors. An example is the optokinetic nystagmus test (Balkema G W et al., 1984, Invest Ophthalmol Vis Sci. 25:795-800; Mitchiner J C et al., 1976, Vision Res. 16:1169-71).

The present invention may also be used in combination with other forms of vision therapy known in the art to improve or restore vision. For example, the use of visual prostheses, which include retinal implants, cortical implants, lateral geniculate nucleus implants, or optic nerve implants. Thus, in addition to genetic modification of surviving retinal neurons using the present methods, the subject being treated may be provided with a visual prosthesis before, at the same time as, or after the molecular method is employed. The effectiveness of visual prosthetics can be improved with training of the individual, thus enhancing the potential impact of the Chop2 transformation of patient cells as contemplated herein. Training methods, such as habituation training characterized by training the subject to recognize (i) varying levels of light and/or pattern stimulation, and/or (ii) environmental stimulation from a common light source or object as would be understood by one skilled in the art; and orientation and mobility training characterized by training the subject to detect visually local objects and move among said objects more effectively than without the training. In fact, any visual stimulation techniques that are typically used in the field of low vision rehabilitation are applicable here.

### EXAMPLES

### Example 1: Generation of Labeled Mutant Chop2 Constructs

Mutations were made on a codon optimized Chop2-GFP fusion protein to create single and double mutations at the L132 (Leucine 132) and T159 (Threonine 159) sites. Several mutants were generated, for example, single mutants such as L132A, L132C, T159A, T159C, and T 159S, and double mutants such as L132C/T159C, L132C/T159S, L132A/T159C, and L132C/T159A. Chop2-GFP transgenes were cloned into a rAAV vector under the control of a CAG promoter using methods known in the art.

### Example 2: In Vitro Analysis of Mutant Chop2 Constructs

The functional properties of each mutant Chop2, or a combination thereof, were first examined in HEK cells. Chop2 constructs were delivered to HEK cells by adenoviral infection, for example. Upon expression of the WT or mutant Chop2, functional WT and mutant ChR2 channels were formed. Measurements of the light sensitivity and other properties of the ChR2 channels were assessed as described herein. The light stimuli (photons/cm²·s at 460 nm) were generated by a xenon arc lamp and attenuated by neutral density filters: ND4.0 (2.8×10¹⁴), ND3.0 (1.4×10¹⁵), ND2.5 (4.8×10¹⁵): ND2.0 (1.6×10¹⁶), ND1.0 (1.3×10¹⁷), ND0 (1.2×10¹⁸). Light evoked currents were measured from wild-type ChR2, T159C, L132C, L132C/T159C, and L132C/T159S. Patch clamp recordings were performed using methods known in the art.

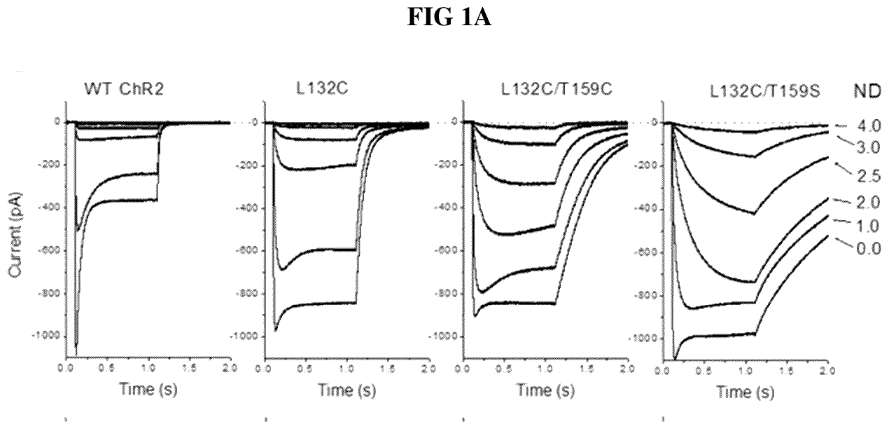

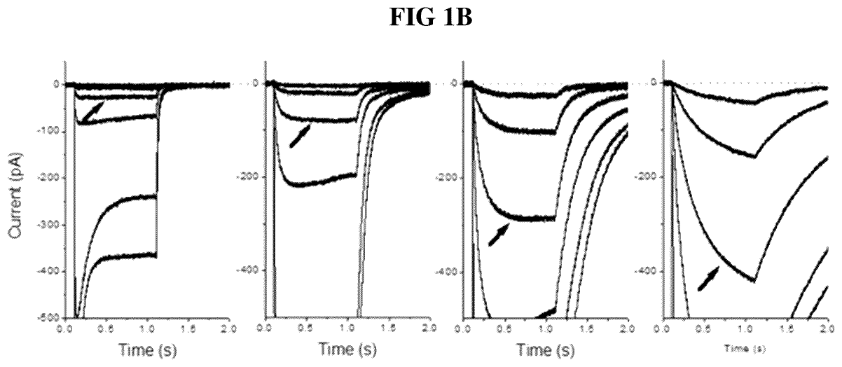

Representative recordings from this experiment comparing light sensitivity between the Chop2 constructs demonstrated that mutations at L132 alone or in combination with mutation at T159 show increased photocurrent in comparison to WT (FIGS. 1A and 1B). FIG. 1B shows the same current traces at a different scale to illustrate the difference in amplitude of the photocurrents between WT ChR2 and ChR2 mutants more clearly. FIG. 1B specifically compares the current traces resulting from light stimulation using the neutral density filter (ND 2.5), equivalent to 4.8×10¹⁵ photos/cm²/s: the traces are designated by the arrows. The amplitude of the photocurrent of the L132C mutant is larger than that of WT: the amplitude of the photocurrent of double mutant L132C/T159C is larger than that of L132C; and the amplitude of the photocurrent of the L132C/T159S mutant larger than L132/T159C. The current traces of the ChR2 mutants, particularly double mutants L132C/T159C and L132C/T159S, also show slower deactivation kinetics when compared to WT and L132C.

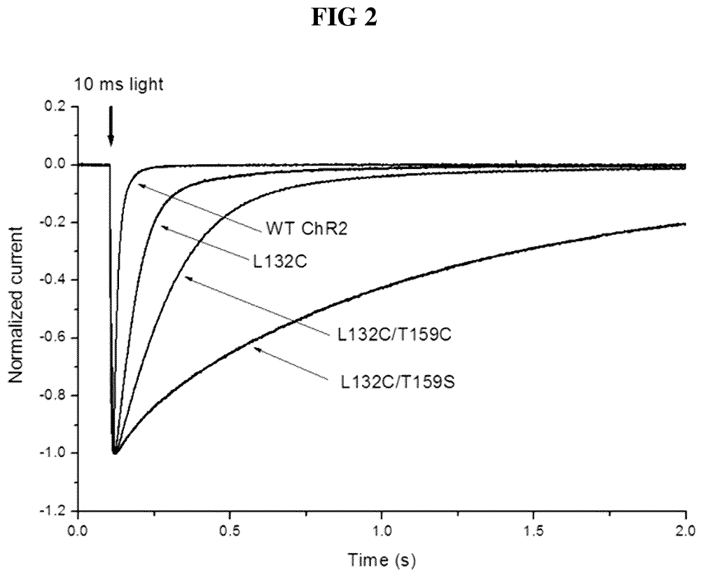

FIG. 2 shows the representative recordings of light-evoked currents from WT ChR2, L132C, L132C/T159C, and L132C/T159S after stimulation by a 10 ms light pulse (1.2×10¹⁸ photons/cm²/s at 460 nm wavelength) to compare the deactivation time course, or decay time course after the light is off. Mutant ChR2 show longer deactivation time courses, with the double mutant L132C/T159S having the longest. Higher light sensitivity, as demonstrated by L132C/T159C and L132C/T159S, may be correlated with slower channel kinetics.

### Example 3: In Vivo Ocular Administration and Analysis of Mutant Chop2 Constructs

AAV2 virus vectors carrying mutant Chop2-GFP constructs driven by CAG promoter were made and injected intravitreally into the eyes of C57BL/6J adult mice. Adult mice were anesthetized by IP injection of ketamine (100 mg/kg) and xylazine (10 mg/kg). Under a dissecting microscope, an incision was made by scissors through the eyelid to expose the sclera. A small perforation was made in the sclera region posterior to the lens with a needle and viral vector suspension of 0.8-1.5 μl at the concentration of approximately 10¹¹ genomic particles/ml was injected into intravitreal space through the hole with a Hamilton syringe with a 32-gauge blunt-ended needle. For each animal, usually only one eye was injected with viral vectors carrying a Chop2 construct, and the other eye was uninjected or injected with control viral vectors carrying GFP alone. Upon expression of the WT or mutant Chop2 of the present invention, functional WT or mutant ChR2 channels were formed utilizing endogenous retinal, and the properties of these ChR2 proteins were assessed as described herein.

ChR2-mediated light responses were examined by using multi-electrode array recordings from whole-mount retinas. Light stimuli (photons/cm²/s) was generated by a 473 nm blue laser and attenuated by neutral density filters: ND0 (6.3×10¹⁶), ND1.0 (7.4×10¹⁵), ND1.5 (2.7×10¹⁵), ND2.0 (7.3×10¹⁴), ND2.5 (3.2×10¹⁴), ND3.0 (8.5×10¹³), ND3.5 (3.8×10¹³), and ND4.0 (9.5×10¹²).

The multielectrode array recordings were based on the procedures reported by Tian and Copenhagen (2003). Briefly, the retina was dissected and placed photoreceptor side down on a nitrocellulose filter paper strip (Millipore Corp., Bedford, MA). The mounted retina was placed in the MEA-60 multielectrode array recording chamber of 30 μm diameter electrodes spaced 200 μm apart (Multi Channel System MCS GmbH, Reutlingen, Germany), with the ganglion cell layer facing the recording electrodes. The retina was continuously perfused in oxygenated extracellular solution at 34° C. during all experiments. The extracellular solution contained (in mM): NaCl, 124; KCl, 2.5; CaCl₂, 2; MgCl₂, 2; NaH₂PO₄, 1.25; NaHCO₃, 26; and glucose, 22 (pH 7.35 with 95% O₂ and 5% CO₂). Recordings were usually started 60 min after the retina was positioned in the recording chamber. The interval between onsets of each light stimulus was 10-15 s. The signals were filtered between 200 Hz (low cut off) and 20 kHz (high cut off). The responses from individual neurons were analyzed using Offline Sorter software (Plexon, Inc., Dallas, TX).

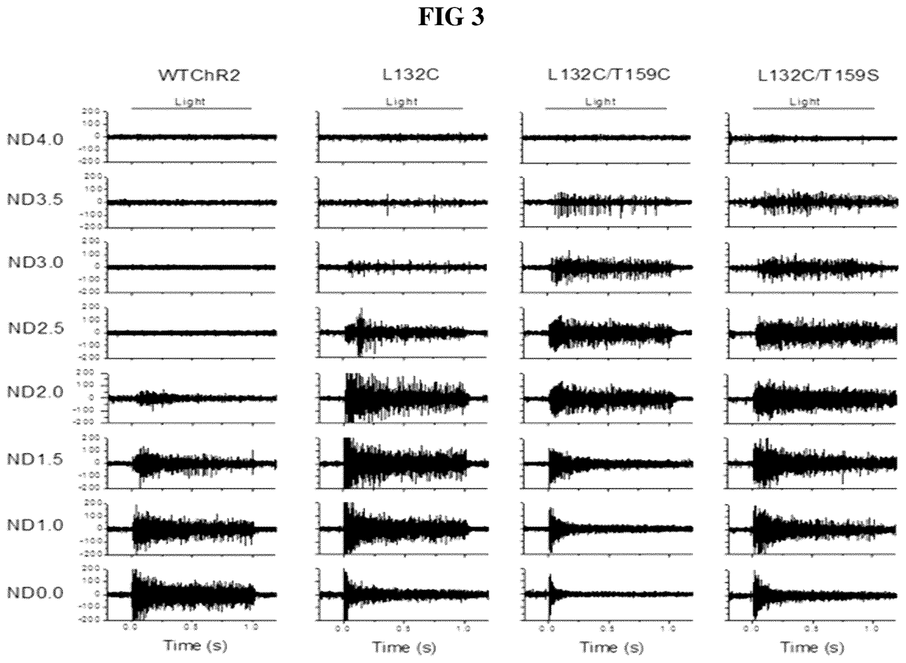

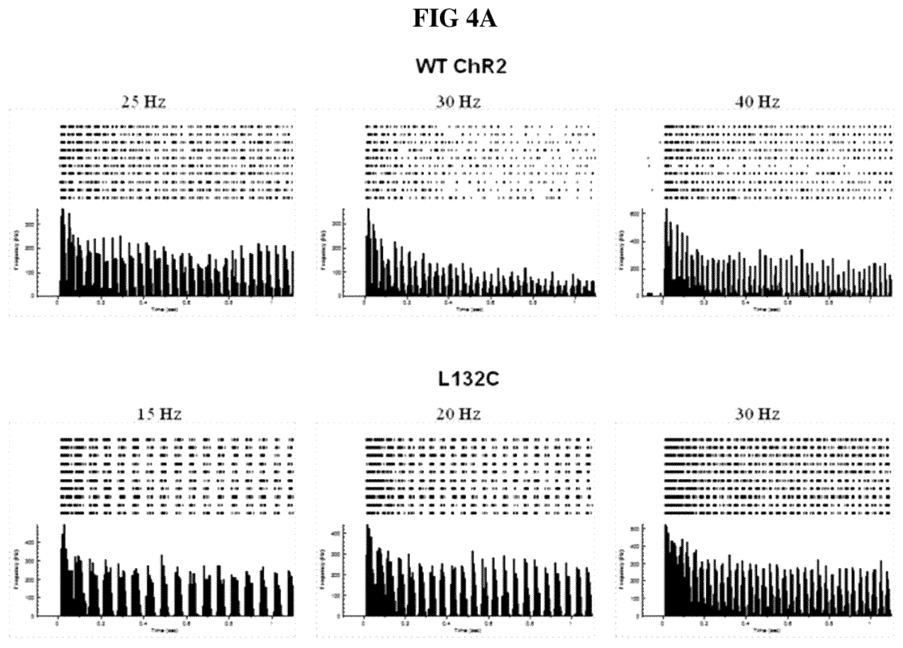

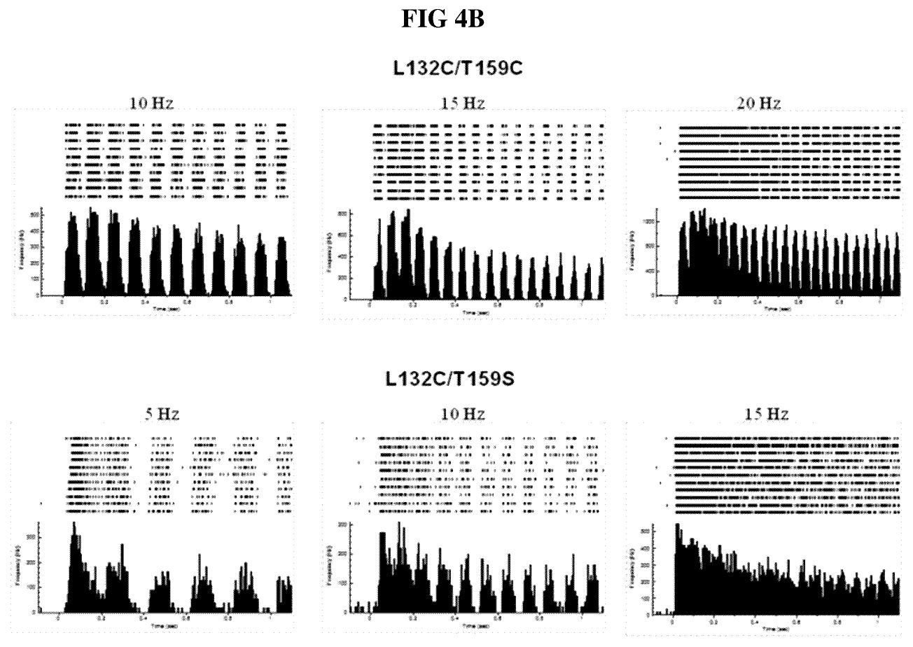

Single mutant Chop2/ChR2 mutants, i.e., L132 and T159C, markedly lower the threshold light intensity that is required to evoke a ChR2-mediated photocurrent. Moreover, several double mutants, including L132C/T159C, L132A/T159C, and L132C/T159S, were found to further increase the photocurrent at low light intensities. Different neutral density filters were used to attenuate the light stimuli to differentiate the light-evoked responses of the Chop2 constructs in low light. Spiking activity of retinal ganglion cells mediated by the mutants of the present invention was observed at the light intensities about 1.5 to 2 log units lower than the light level that is required to elicit the spiking activity with wild-type ChR2 (FIG. 3). Specifically, WT ChR2 exhibited did not exhibit any spiking activity in response to light stimuli with neutral density filter 2.5 (3.2×10¹⁴ photons/cm²/s) while ChR2 mutants (L132C, L132C/T159C, and L132C/T159S) demonstrate spiking activity. In fact, the ChR2 mutants still exhibited spiking activity in response to light with neutral density filters 3.0 and 3.5. Therefore, ChR2 mutants of the present invention possess higher light sensitivity and, thus, a markedly lower threshold light intensity that is required to elicit a ChR2-mediated photocurrent. Moreover, ChR2 double mutants possess a higher light sensitivity than single mutants, i.e. L132C. In addition, the spike firing of retinal ganglion cells expressing L132C/T159C and L132/T159S could follow a light flicker frequency of up to 15 Hz and 5 Hz, respectively (FIG. 4).

The L132C/T159A mutant shows high light sensitivity, probably the most light sensitive among these mutants, but it also shows extremely slow off-rate (the channel continue open for many many sends after light off). Interestingly, it can be turned off more quickly using a light with long-wavelengths, such as yellow light. The L132C/T159A mutant (encoded by SEQ ID NOs: 24 and 25) demonstrates significant potential.

Given the trade-off between light sensitivity and channel kinetics, Chop2/ChR2 mutants that demonstrate a balance between light sensitivity and channel kinetics, such as L132C/T159C or L132C/T159S, may be suitable for the application of vision restoration.

### Example 4: Analysis of Mutant Chop2 Constructs in Mouse Models of Disease

Mouse models of degenerative ocular diseases are known in the art. For example, homozygous rd1 (rd1/rd1) mice are a commonly used photoreceptor degeneration model. Rd1 mice carry a null mutation in a cyclic GMP phosphodiesterase, PDE6, similar to some forms of retinitis pigmentosa in humans. Other well-established mouse models of ocular disease that may be of particular interest to demonstrate ChR2 mutant safety and efficacy include rds (also known as Prph^{Rd2}), rd3, rd4, rd5, rd6, rd7, rd8, rd9, Pde6b^{rd10}, or cpfl1 mice.

The Chop2-GFP constructs of the present invention can be injected intravitreally into the eyes of newborn (P1) or adult mice at 2-12 months of age. GFP signal can be observed in the Chop2-GFP-injected retinas, to determine the levels of ChR2 expression or expression in particular populations of cells, such as the retinal ganglion cells. Mutant Chop2-GFP expression can be monitored for a predetermined amount of time, i.e. 3-6 months, or 1 year after viral injection. Patch-clamp and multichannel array recordings can be performed using the methods known in the art and described herein to measure the light-evoked responses of mutant Chop2-GFP-expressing cells in vivo.

Additional techniques and tests are well-established in the art to test for the restoration of light sensitivity or vision. Visual evoked potentials from the Chop2-GFP expressing cells or visual cortex can be examined, as described in PCT publication WO 2007/131180. Other tests include behavioral assessments of the visual acuity in the mice, i.e., virtual optomotor test and visual water maze.

### Example 5: Analysis of Long-Term Expression and Safety of Administration of Mutant Chop2 Constructs to Retinal Neurons

Neurotoxicity was assessed in C57BL/6J adult mice injected with Chop2 constructs of the present invention. The expression safety of Chop2 mutants in the retina was assessed by immunostaining and cell counting after exposure to strong blue light for two weeks. None of the mice were found to exhibit symptoms of neurotoxicity for up to two months after injection.

Additional ongoing studies are evaluating the long-term expression and safety of Chop2/ChR2 mutants of the invention in retinal neurons.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. An expression vector comprising a polynucleotide encoding a polypeptide comprising an amino acid sequence of SEQ ID NO: 26 in which the amino acid at position 132 of SEQ ID NO: 26 is cysteine (C) or alanine (A), and the amino acid at position 159 of SEQ ID NO: 26 is cysteine (C), serine (S), or alanine (A).

2. The expression vector of claim 1, wherein the polypeptide comprises a cysteine (C) at positions 132 and 159 of SEQ ID NO: 26.

3. The expression vector of claim 2, wherein the polypeptide comprises the amino acid sequence of SEQ ID NO: 16 and the polynucleotide comprises the nucleic acid sequence of SEQ ID NO: 15.

4. The expression vector of claim 1, wherein the polypeptide comprises a cysteine (C) at position 132 and a serine (S) at position 159 of SEQ ID NO: 26.

5. The expression vector of claim 4, wherein the polypeptide comprises the amino acid sequence of SEQ ID NO: 19 and the polynucleotide comprises the nucleic acid sequence of SEQ ID NO: 18.

6. The expression vector of claim 1, wherein the polypeptide comprises a cysteine (C) at position 132 and an alanine (A) at position 159 of SEQ ID NO: 26.

7. The expression vector of claim 6, wherein the polypeptide comprises the amino acid sequence of SEQ ID NO: 25 and the polynucleotide comprises the nucleic acid sequence of SEQ ID NO: 24.

8. The expression vector of claim 1, wherein the polypeptide comprises an alanine (A) at position 132 and a cysteine (C) at position 159 of SEQ ID NO: 26.

9. The expression vector of claim 8, wherein the polypeptide comprises the amino acid sequence of SEQ ID NO: 22 and the polynucleotide comprises the nucleic acid sequence of SEQ ID NO: 21.

10. The expression vector of claim 1, wherein the polypeptide comprises an alanine (A) at position 132 and a serine (S) at position 159 of SEQ ID NO: 26.

11. The expression vector of claim 1, wherein the polypeptide comprises an alanine (A) at position 132 and an alanine (A) at position 159 of SEQ ID NO: 26.

12. The expression vector of claim 1, wherein the expression vector is an adeno-associated virus (AAV) vector.

13. The expression vector of claim 12, wherein the AAV vector is an AAV2 vector.

14. A method of improving or restoring vision in a subject, the method comprising administering to the subject the expression vector of claim 1.

15. A method of improving or restoring vision in a subject, the method comprising administering to the subject the expression vector of claim 12.

16. A method of improving or restoring vision in a subject, the method comprising administering to the subject the expression vector of claim 13.

17. The method of claim 14, wherein the subject either has normal vision or the subject is suffering from an ocular disease.

18. The method of claim 15, wherein the subject either has normal vision or the subject is suffering from an ocular disease.

19. The method of claim 16, wherein the subject either has normal vision or the subject is suffering from an ocular disease.

20. The method of claim 17, wherein the subject is suffering from an ocular disease and the ocular disease is macular degeneration or retinitis pigmentosa.

21. The method of claim 18, wherein the subject is suffering from an ocular disease and the ocular disease is macular degeneration or retinitis pigmentosa.

22. The method of claim 19, wherein the subject is suffering from an ocular disease and the ocular disease is macular degeneration or retinitis pigmentosa.


SEQUENCE LISTING (26 sequences)

SEQ ID NO: 1 - DNA, 3585 bp, Chlamydomonas reinhardtii
  source 1..3585: mol_type = other DNA
gcagcaccatacttgacatctgtcgccaagcaagcattaaacatggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtcaacaagggcaccggcaagtacgcctcccgcgagtccttcctggtcatgcgcgacaagatgaaggagaagggcattgacgtgcgcgcctctctggacaacagcaaggaggtggagcaggagcaggccgccagggctgccatgatgatgatgaacggcaatggcatgggtatgggaatgggaatgaacggcatgaacggaatgggcggtatgaacgggatggctggcggcgccaagcccggcctggagctcactccgcagctacagcccggccgcgtcatcctggcggtgccggacatcagcatggttgacttcttccgcgagcagtttgctcagctatcggtgacgtacgagctggtgccggccctgggcgctgacaacacactggcgctggttacgcaggcgcagaacctgggcggcgtggactttgtgttgattcaccccgagttcctgcgcgaccgctctagcaccagcatcctgagccgcctgcgcggcgcgggccagcgtgtggctgcgttcggctgggcgcagctggggcccatgcgtgacctgatcgagtccgcaaacctggacggctggctggagggcccctcgttcggacagggcatcctgccggcccacatcgttgccctggtggccaagatgcagcagatgcgcaagatgcagcagatgcagcagattggcatgatgaccggcggcatgaacggcatgggcggcggtatgggcggcggcatgaacggcatgggcggcggcaacggcatgaacaacatgggcaacggcatgggcggcggcatgggcaacggcatgggcggcaatggcatgaacggaatgggtggcggcaacggcatgaacaacatgggcggcaacggaatggccggcaacggaatgggcggcggcatgggcggcaacggtatgggtggctccatgaacggcatgagctccggcgtggtggccaacgtgacgccctccgccgccggcggcatgggcggcatgatgaacggcggcatggctgcgccccagtcgcccggcatgaacggcggccgcctgggtaccaacccgctcttcaacgccgcgccctcaccgctcagctcgcagctcggtgccgaggcaggcatgggcagcatgggaggcatgggcggaatgagcggaatgggaggcatgggtggaatggggggcatgggcggcgccggcgccgccacgacgcaggctgcgggcggcaacgcggaggcggagatgctgcagaatctcatgaacgagatcaatcgcctgaagcgcgagcttggcgagtaaaaggctggaggccggtactgcgatacctgcgagctcgcgcgcctgactcgtcgtacacacggctcaggagcacgcgcgcgtggacttctcaacctgtgtgcaacgtatctagagcggcctgtgcgcgaccgtccgtgagcattccggtgcgatcttcccgccttcgcaccgcaagttcccttcctggccctgctgcgcctgacgcatcgtccgaacggaagggcggcttgatcagtaaagcattgaagactgaagtcgtgcgaccgtagtgctatggctctgcacgtaagtgggcgctgccctgcttactacgcattgcccaagactgcttccttttggtggccgaggccctggtcccacatcattcatttgcataacgtactgtttagttacatacgctttgcttaacctcgacaattgcaacatgggctgagagtccgtacggcggctatggacgaaggtgttatcggatgtgattaggaatctcggttgaaaggcttcgagaaagtgagcttcatctgtggcttctgttggggtcatcaagaagaacgacggtaaggcaaacgaggtaaaagtggcacgtctttgtgcacaacgggcccgtggagagtgggggagtgcatgtgtgcggtcctaacacgcgagtgcaaagcgggcttttctggagctgggttacggtctggctcggcaactgctctgtgttttaaccacagcttcggaagtctgggtatgttttgttggcagaaacatttgggtaacttgagggtgattcgtctggagtcggacaacatggctgccgtccgtgtgcagggacggtaatcaatgagctggagctgtgatgctcaccacacgttgcatacccctgcttacaaaaacactttgatgtcgtggccaaactatgcgtgagcaaagagttaaagaggcatgagtgcatggttgcggacgtgcgcaacaattgcatcaagtatttgacgccttcaagccaacaagtgcgcgcgcggcaacttgattaacacgccggacgcagtggtgggggcgtgtacagtgtttatgagctgccattctgcgatccgtagtgttaggttgcgtgtgacgccgcgcggctgtgggcccttacatggagagttgggtgcttcaccacacggttggcgccgctgaagggtgtgctatgttttggtaaagccggggccctgaagaccgcaaccgtagaaccgtactgaaagggtgtcagcccggggtaactggatgccctgggacatagctattaatgttgaagtgaagccgtcaagccgagtgccgtgcgccgctgtatcaccaaggcccgtccta

SEQ ID NO: 2 - protein, 737 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGKYASRESFLVMRDKMKEKGIDVRASLDNSKEVEQEQAARAAMMMMNGNGMGMGMGMNGMNGMGGMNGMAGGAKPGLELTPQLQPGRVILAVPDISMVDFFREQFAQLSVTYELVPALGADNTLALVTQAQNLGGVDFVLIHPEFLRDRSSTSILSRLRGAGQRVAAFGWAQLGPMRDLIESANLDGWLEGPSFGQGILPAHIVALVAKMQQMRKMQQMQQIGMMTGGMNGMGGGMGGGMNGMGGGNGMNNMGNGMGGGMGNGMGGNGMNGMGGGNGMNNMGGNGMAGNGMGGGMGGNGMGGSMNGMSSGVVANVTPSAAGGMGGMMNGGMAAPQSPGMNGGRLGTNPLFNAAPSPLSSQLGAEAGMGSMGGMGGMSGMGGMGGMGGMGGAGAATTQAAGGNAEAEMLQNLMNEINRLKRELGE

SEQ ID NO: 3 - DNA, 2241 bp, Chlamydomonas reinhardtii
  source 1..2241: mol_type = other DNA
gcatctgtcgccaagcaagcattaaacatggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtcaacaagggcaccggcaagtacgcctcccgcgagtccttcctggtcatgcgcgacaagatgaaggagaagggcattgacgtgcgcgcctctctggacaacagcaaggaggtggagcaggagcaggccgccagggctgccatgatgatgatgaacggcaatggcatgggtatgggaatgggaatgaacggcatgaacggaatgggcggtatgaacgggatggctggcggcgccaagcccggcctggagctcactccgcagctacagcccggccgcgtcatcctggcggtgccggacatcagcatggttgacttcttccgcgagcagtttgctcagctatcggtgacgtacgagctggtgccggccctgggcgctgacaacacactggcgctggttacgcaggcgcagaacctgggcggcgtggactttgtgttgattcaccccgagttcctgcgcgaccgctctagcaccagcatcctgagccgcctgcgcggcgcgggccagcgtgtggctgcgttcggctgggcgcagctggggcccatgcgtgacctgatcgagtccgcaaacctggacggctggctggagggcccctcgttcggacagggcatcctgccggcccacatcgttgccctggtggccaagatgcagcagatgcgcaagatgcagcagatgcagcagattggcatgatgaccggcggcatgaacggcatgggcggcggtatgggcggcggcatgaacggcatgggcggcggcaacggcatgaacaacatgggcaacggcatgggcggcggcatgggcaacggcatgggcggcaatggcatgaacggaatgggtggcggcaacggcatgaacaacatgggcggcaacggaatggccggcaacggaatgggcggcggcatgggcggcaacggtatgggtggctccatgaacggcatgagctccggcgtggtggccaacgtgacgccctccgccgccggcggcatgggcggcatgatgaacggcggcatggctgcgccccagtcgcccggcatgaacggcggccgcctgggtaccaacccgctcttcaacgccgcgccctcaccgctcagctcgcagctcggtgccgaggcaggcatgggcagcatgggaggcatgggcggaatgagcggaatgggaggcatgggtggaatggggggcatgggcggcgccggcgccgccacgacgcaggctgcgggcggcaacgcggaggcggagatgctgcagaatctcatgaacgagatcaatcgcctgaagcgcgagcttggcgagtaa

SEQ ID NO: 4 - protein, 737 aa, Chlamydomonas reinhardtii; identical to SEQ ID NO: 2

SEQ ID NO: 5 - DNA, 3599 bp, Chlamydomonas reinhardtii
  source 1..3599: mol_type = other DNA
ttgacatctgtcgccaagcaagcattaaacatggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtcaacaagggcaccggcaagtacgcctcccgcgagtccttcctggtcatgcgcgacaagatgaaggagaagggcattgacgtgcgcgcctctctggacaacagcaaggaggtggagcaggagcaggccgccagggctgccatgatgatgatgaacggcaatggcatgggtatgggaatgggaatgaacggcatgaacggaatgggcggtatgaacgggatggctggcggcgccaagcccggcctggagctcactccgcagctacagcccggccgcgtcatcctggcggtgccggacatcagcatggttgacttcttccgcgagcagtttgctcagctatcggtgacgtacgagctggtgccggccctgggcgctgacaacacactggcgctggttacgcaggcgcagaacctgggcggcgtggactttgtgttgattcaccccgagttcctgcgcgaccgctctagcaccagcatcctgagccgcctgcgcggcgcgggccagcgtgtggctgcgttcggctgggcgcagctggggcccatgcgtgacctgatcgagtccgcaaacctggacggctggctggagggcccctcgttcggacagggcatcctgccggcccacatcgttgccctggtggccaagatgcagcagatgcgcaagatgcagcagatgcagcagattggcatgatgaccggcggcatgaacggcatgggcggcggtatgggcggcggcatgaacggcatgggcggcggcaacggcatgaacaacatgggcaacggcatgggcggcggcatgggcaacggcatgggcggcaatggcatgaacggaatgggtggcggcaacggcatgaacaacatgggcggcaacggaatggccggcaacggaatgggcggcggcatgggcggcaacggtatgggtggctccatgaacggcatgagctccggcgtggtggccaacgtgacgccctccgccgccggcggcatgggcggcatgatgaacggcggcatggctgcgccccagtcgcccggcatgaacggcggccgcctgggtaccaacccgctcttcaacgccgcgccctcaccgctcagctcgcagctcggtgccgaggcaggcatgggcagcatgggaggcatgggcggaatgagcggaatgggaggcatgggtggaatggggggcatgggcggcgccggcgccgccacgacgcaggctgcgggcggcaacgcggaggcggagatgctgcagaatctcatgaacgagatcaatcgcctgaagcgcgagcttggcgagtaaaaggctggaggccggtactgcgatacctgcgagctcgcgcgcctgactcgtcgtacacacggctcaggagcacgcgcgcgtggacttctcaacctgtgtgcaacgtatctagagcggcctgtgcgcgaccgtccgtgagcattccggtgcgatcttcccgccttcgcaccgcaagttcccttcctggccctgctgcgcctgacgcatcgtccgaacggaagggcggcttgatcagtaaagcattgaagactgaagtcgtgcgaccgtagtgctatggctctgcacgtaagtgggcgctgccctgcttactacgcattgcccaagactgcttccttttggtggccgaggccctggtcccacatcattcatttgcataacgtactgtttagttacatacgctttgcttaacctcgacaattgcaacatgggctgagagtccgtacggcggctatggacgaaggtgttatcggatgtgattaggaatctcggttgaaaggcttcgagaaagtgagcttcttctgtggcttctgttggggtcatcaagaagaacgacggtaaggcaaacgaggtaaaagtggcacgtctttgtgcacaacgggcccgtggagagtgggggagtgcatgtgtgcggtcctaacacgcgagtgcaaagcgggcttttctggagctgggttacggtctggctcggcaactgctctgtgttttaaccacagcttcggaagtctgggtatgttttgttggcagaaacatttgggtaacttgagggtgattcgtctggagtcggacaacatggctgccgtccgtgtgcagggacggtaatcaatgaagctgaagctgtgatgctcaccacacgttgcatacccctgcttacaaaaacactttgatgtcgtggccaaactatgcgtgagcaaagagttaaagaggcatgagtgcatggttgcggacgtgcgcaacaattgcatcaagtatttgacgccttcaagccaacaagtgcgcgcgcggcaacttgattaacacgccggacgcagtggtgggggcgtgtacagtgtttatgagctgccattctgcgatccgtagtgttaggttgcgtgtgacgccgcgcggctgtgggcccttacatggagagttgggtgcttcaccacacggttggcgccgctgaagggtgtgctatgttttggtaaagccggggccctgaagaccgcaaccgtagaaccgtactgaaagggtgtcagcccggggtaactggatgccctgggacatagctattaatgttgaagtgaagccgtcaagccgagtgccgtgcgccgctgtatcaccaaggcccgtccaaaaaaaaaaaaaaaaaaaaaaaaaaa

SEQ ID NO: 6 - protein, 737 aa, Chlamydomonas reinhardtii; identical to SEQ ID NO: 2

SEQ ID NO: 7 - DNA, 2448 bp, Chlamydomonas reinhardtii
  source 1..2448: mol_type = other DNA
catctgtcgccaagcaagcattaaacatggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtcaacaagggcaccggcaagtacgcctcccgcgagtccttcctggtcatgcgcgacaagatgaaggagaagggcattgacgtgcgcgcctctctggacaacagcaaggaggtggagcaggagcaggccgccagggctgccatgatgatgatgaacggcaatggcatgggtatgggaatgggaatgaacggcatgaacggaatgggcggtatgaacgggatggctggcggcgccaagcccggcctggagctcactccgcagctacagcccggccgcgtcatcctggcggtgccggacatcagcatggttgacttcttccgcgagcagtttgctcagctatcggtgacgtacgagctggtgccggccctgggcgctgacaacacactggcgctggttacgcaggcgcagaacctgggcggcgtggactttgtgttgattcaccccgagttcctgcgcgaccgctctagcaccagcatcctgagccgcctgcgcggcgcgggccagcgtgtggctgcgttcggctgggcgcagctggggcccatgcgtgacctgatcgagtccgcaaacctggacggctggctggagggcccctcgttcggacagggcatcctgccggcccacatcgttgccctggtggccaagatgcagcagatgcgcaagatgcagcagatgcagcagattggcatgatgaccggcggcatgaacggcatgggcggcggtatgggcggcggcatgaacggcatgggcggcggcaacggcatgaacaacatgggcaacggcatgggcggcggcatgggcaacggcatgggcggcaatggcatgaacggaatgggtggcggcaacggcatgaacaacatgggcggcaacggaatggccggcaacggaatgggcggcggcatgggcggcaacggtatgggtggctccatgaacggcatgagctccggcgtggtggccaacgtgacgccctccgccgccggcggcatgggcggcatgatgaacggcggcatggctgcgccccagtcgcccggcatgaacggcggccgcctgggtaccaacccgctcttcaacgccgcgccctcaccgctcagctcgcagctcggtgccgaggcaggcatgggcagcatgggaggcatgggcggaatgagcggaatgggaggcatgggtggaatggggggcatgggcggcgccggcgccgccacgacgcaggctgcgggcggcaacgcggaggcggagatgctgcagaatctcatgaacgagatcaatcgcctgaagcgcgagcttggcgagtaaaaggctggaggccggtactgcgatacctgcgagctcgcgcgcctgactcgtcgtacacacggctcaggagcacgcgcgcgtggacttctcaacctgtgtgcaacgtatctagagcggcctgtgcgcgaccgtccgtgagcattccggtgcgatcttcccgccttcgcaccgcaagttcccttcctggccctgctgcgcctgacgcatc

SEQ ID NO: 8 - protein, 737 aa, Chlamydomonas reinhardtii; identical to SEQ ID NO: 2

SEQ ID NO: 9 - DNA, 3033 bp, synthetic construct
  misc_feature 1..3033: note = ChR2 mutant
  source 1..3033: mol_type = other DNA
atggattaccctgtggcccggtccctgattgtaagataccccaccgatctgggcaatggaaccgtgtgcatgcccagaggacaatgctactgcgaggggtggctgaggagccggggcactagtatcgaaaaaaccatcgctatcaccctccagtgggtagtgttcgctctgtccgtagcctgtctcggctggtatgcataccaagcctggagggctacctgtgggtgggaggaagtatacgtggccctgatcgagatgatgaagtccatcatcgaggctttccatgagttcgactccccagccacactctggctcagcagtgggaatggcgtagtgtggatgagatatggagagtggctgctgacctgtcccgtcctgctcattcatctgtccaatctgaccgggctgaaagatgactactccaagagaacaatgggactgctggtgagtgacgtggggtgtattgtgtggggagccacctccgccatgtgcactggatggaccaagatcctctttttcctgatttccctctcctatgggatgtatacatacttccacgccgctaaggtgtatattgaggccttccacactgtacctaaaggcatctgtagggagctcgtgcgggtgatggcatggaccttctttgtggcctgggggatgttccccgtgctgttcctcctcggcactgagggatttggccacattagtccttacgggtccgcaattggacactccatcctggatctgattgccaagaatatgtggggggtgctgggaaattatctgcgggtaaagatccacgagcatatcctgctgtatggcgatatcagaaagaagcagaaaatcaccattgctggacaggaaatggaggtggagacactggtagcagaggaggaggacgggaccgcggtcgccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgcctgcaggagaagaagtcatgcagccagcgcatggccgaattccggcaatactgttggaacccggacactgggcagatgctgggccgcaccccagcccggtgggtgtggatcagcctgtactatgcagctttctacgtggtcatgactgggctctttgccttgtgcatctatgtgctgatgcagaccattgatccctacacccccgactaccaggaccagttaaagtcaccgggggtaaccttgagaccggatgtgtatggggaaagagggctgcagatttcctacaacatctctgaaaacagctctagacaggcccagatcaccggacgtccggagactgagacattgccaccggtggactacgggggggccctgagcgctgtgggcagagaactcctgttcgtgacaaatccagtcgtggtgaacggctccgtactcgtacccgaggatcagtgctattgcgcaggatggatcgagagcagaggcacaaacggcgcacagactgcatccaacgtgctccagtggttggccgcaggcttttccattctcctgctcatgttttacgcctaccagacttggaagtccacatgtggctgggaggaaatctacgtgtgtgcaatcgaaatggtgaaggtgatcctggagtttttcttcgaatttaaaaacccaagcatgctgtacctggctactggccacagagtgcagtggctgcggtatgccgaatggctgctgacttgcccagtgatttgcatccacctgtccaacctgactgggctgtctaacgattacagtaggagaacaatgggactgctcgtatccgacatcggcactatcgtatggggcgcaactagtgccatggccactggatacgtgaaagtgatcttcttctgcctgggactctgctacggagcaaacacattttttcatgccgcaaaagcatatatcgaggggtatcataccgtcccaaagggccggtgtagacaagtggtgactggcatggcttggctgttcttcgtgtcctgggggatgtttcccatcctctttatcctgggcccagaaggcttcggggtgctgagtgtgtatggcagtaccgtaggacacactatcattgacctgatgagcaaaaactgctgggggctgctcggccactacctgagagtactcatccacgagcatatcctgattcatggcgatatccggaaaactaccaagctcaatatcgggggcaccgagattgaagtggagacactcgtggaggacgaggccgaggccggagcagtgaacaaaggcactggcaagtatgcctccagagaatcctttctggtgatgcgggacaaaatgaaggagaaaggcattgatgtacggtgcagtaatgccaaagccgtcgagactgatgtgtag

SEQ ID NO: 10 - protein, 1010 aa, synthetic construct
  REGION 1..1010: note = ChR2 mutant
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDGTAVATMVSKGEELIKENMHMKLYMEGTVNNHHFKCTSEGEGKPYEGTQTMRIKVVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTEMLYPADGGLEGRADMALKLVGGGHLICNLKTTYRSKKPAKNLKMPGVYYVDRRLERIKEADKETYVEQHEVAVARYCDLPSKLGHKLNCLQEKKSCSQRMAEFRQYCWNPDTGQMLGRTPARWVWISLYYAAFYVVMTGLFALCIYVLMQTIDPYTPDYQDQLKSPGVTLRPDVYGERGLQISYNISENSSRQAQITGRPETETLPPVDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVICIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGKYASRESFLVMRDKMKEKGIDVRCSNAKAVETDV

SEQ ID NO: 11 - DNA, 3033 bp, synthetic construct
  misc_feature 1..3033: note = ChR2 mutant
  source 1..3033: mol_type = other DNA
atggattaccctgtggcccggtccctgattgtaagataccccaccgatctgggcaatggaaccgtgtgcatgcccagaggacaatgctactgcgaggggtggctgaggagccggggcactagtatcgaaaaaaccatcgctatcaccctccagtgggtagtgttcgctctgtccgtagcctgtctcggctggtatgcataccaagcctggagggctacctgtgggtgggaggaagtatacgtggccctgatcgagatgatgaagtccatcatcgaggctttccatgagttcgactccccagccacactctggctcagcagtgggaatggcgtagtgtggatgagatatggagagtggctgctgacctgtcccgtcctgctcattcatctgtccaatctgaccgggctgaaagatgactactccaagagaacaatgggactgctggtgagtgacgtggggtgtattgtgtggggagccacctccgccatgtgcactggatggaccaagatcctctttttcctgatttccctctcctatgggatgtatacatacttccacgccgctaaggtgtatattgaggccttccacactgtacctaaaggcatctgtagggagctcgtgcgggtgatggcatggaccttctttgtggcctgggggatgttccccgtgctgttcctcctcggcactgagggatttggccacattagtccttacgggtccgcaattggacactccatcctggatctgattgccaagaatatgtggggggtgctgggaaattatctgcgggtaaagatccacgagcatatcctgctgtatggcgatatcagaaagaagcagaaaatcaccattgctggacaggaaatggaggtggagacactggtagcagaggaggaggacgggaccgcggtcgccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgcctgcaggagaagaagtcatgcagccagcgcatggccgaattccggcaatactgttggaacccggacactgggcagatgctgggccgcaccccagcccggtgggtgtggatcagcctgtactatgcagctttctacgtggtcatgactgggctctttgccttgtgcatctatgtgctgatgcagaccattgatccctacacccccgactaccaggaccagttaaagtcaccgggggtaaccttgagaccggatgtgtatggggaaagagggctgcagatttcctacaacatctctgaaaacagctctagacaggcccagatcaccggacgtccggagactgagacattgccaccggtggactacgggggggccctgagcgctgtgggcagagaactcctgttcgtgacaaatccagtcgtggtgaacggctccgtactcgtacccgaggatcagtgctattgcgcaggatggatcgagagcagaggcacaaacggcgcacagactgcatccaacgtgctccagtggttggccgcaggcttttccattctcctgctcatgttttacgcctaccagacttggaagtccacatgtggctgggaggaaatctacgtgtgtgcaatcgaaatggtgaaggtgatcctggagtttttcttcgaatttaaaaacccaagcatgctgtacctggctactggccacagagtgcagtggctgcggtatgccgaatggctgctgacttgcccagtgattctgatccacctgtccaacctgactgggctgtctaacgattacagtaggagaacaatgggactgctcgtatccgacatcggcactatcgtatggggcgcaactagtgccatggccactggatacgtgaaagtgatcttcttctgcctgggactctgctacggagcaaacacattttttcatgccgcaaaagcatatatcgaggggtatcataccgtcccaaagggccggtgtagacaagtggtgactggcatggcttggctgttcttcgtgtcctgggggatgtttcccatcctctttatcctgggcccagaaggcttcggggtgctgagtgtgtatggcagtaccgtaggacacactatcattgacctgatgagcaaaaactgctgggggctgctcggccactacctgagagtactcatccacgagcatatcctgattcatggcgatatccggaaaactaccaagctcaatatcgggggcaccgagattgaagtggagacactcgtggaggacgaggccgaggccggagcagtgaacaaaggcactggcaagtatgcctccagagaatcctttctggtgatgcgggacaaaatgaaggagaaaggcattgatgtacggtgcagtaatgccaaagccgtcgagactgatgtgtag

SEQ ID NO: 12 - protein, 1010 aa, synthetic construct
  REGION 1..1010: note = ChR2 mutant construct
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDGTAVATMVSKGEELIKENMHMKLYMEGTVNNHHFKCTSEGEGKPYEGTQTMRIKVVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTEMLYPADGGLEGRADMALKLVGGGHLICNLKTTYRSKKPAKNLKMPGVYYVDRRLERIKEADKETYVEQHEVAVARYCDLPSKLGHKLNCLQEKKSCSQRMAEFRQYCWNPDTGQMLGRTPARWVWISLYYAAFYVVMTGLFALCIYVLMQTIDPYTPDYQDQLKSPGVTLRPDVYGERGLQISYNISENSSRQAQITGRPETETLPPVDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGKYASRESFLVMRDKMKEKGIDVRCSNAKAVETDV

SEQ ID NO: 13 - protein, 315 aa, synthetic construct
  REGION 1..315: note = ChR2 mutant construct
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVICIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 14 - protein, 315 aa, synthetic construct
  REGION 1..315: note = ChR2 mutant construct
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGCIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 15 - DNA, 945 bp, synthetic construct
  misc_feature 1..945: note = ChR2 mutant construct
  source 1..945: mol_type = other DNA
atggactacgggggggctctgtctgctgtcgggagggaactgctgtttgtgactaaccctgtcgtcgtgaacgggagtgtgctggtccctgaggaccagtgctactgtgccggctggatcgaatcacgcggaaccaacggggcccagacagctagcaatgtgctgcagtggctggccgctgggtttagtatcctgctgctgatgttctacgcctatcagacttggaagtcaacctgcggctgggaggaaatctacgtgtgcgctattgagatggtgaaagtgatcctggagttcttcttcgagttcaagaacccaagcatgctgtacctggctactggacaccgagtgcagtggctgagatatgcagaatggctgctgacatgccccgtcatctgcattcacctgtccaacctgacaggcctgagcaatgactactccaggagaactatgggactgctggtgtccgacatcggctgcattgtctggggagcaacttctgctatggcaaccggatacgtgaaggtcatctttttctgcctggggctgtgctatggcgcaaatacctttttccacgcagccaaggcctacattgaggggtatcataccgtgccaaaaggccggtgccgacaggtggtcacaggaatggcttggctgtttttcgtctcttggggaatgtttcccatcctgttcattctggggcctgaagggttcggcgtgctgtctgtctacggaagtacagtggggcatactatcattgacctgatgtccaaaaactgttggggcctgctgggacactatctgagagtgctgatccacgagcatatcctgattcatggcgatattcggaagaccacaaaactgaatatcggcggaaccgagattgaagtggaaacactggtggaagacgaggctgaggctggggctgtgaacaaggggactggcaaa

SEQ ID NO: 16 - protein, 315 aa, synthetic construct
  REGION 1..315: note = mutated Chop2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVICIHLSNLTGLSNDYSRRTMGLLVSDIGCIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 17 - protein, 315 aa, synthetic construct
  REGION 1..315: note = mutated Chop2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGSIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 18 - DNA, 945 bp, synthetic construct
  misc_feature 1..945: note = mutated Chop2
  source 1..945: mol_type = other DNA
atggactacgggggggctctgtctgctgtcgggagggaactgctgtttgtgactaaccctgtcgtcgtgaacgggagtgtgctggtccctgaggaccagtgctactgtgccggctggatcgaatcacgcggaaccaacggggcccagacagctagcaatgtgctgcagtggctggccgctgggtttagtatcctgctgctgatgttctacgcctatcagacttggaagtcaacctgcggctgggaggaaatctacgtgtgcgctattgagatggtgaaagtgatcctggagttcttcttcgagttcaagaacccaagcatgctgtacctggctactggacaccgagtgcagtggctgagatatgcagaatggctgctgacatgccccgtcatctgcattcacctgtccaacctgacaggcctgagcaatgactactccaggagaactatgggactgctggtgtccgacatcggcagcattgtctggggagcaacttctgctatggcaaccggatacgtgaaggtcatctttttctgcctggggctgtgctatggcgcaaatacctttttccacgcagccaaggcctacattgaggggtatcataccgtgccaaaaggccggtgccgacaggtggtcacaggaatggcttggctgtttttcgtctcttggggaatgtttcccatcctgttcattctggggcctgaagggttcggcgtgctgtctgtctacggaagtacagtggggcatactatcattgacctgatgtccaaaaactgttggggcctgctgggacactatctgagagtgctgatccacgagcatatcctgattcatggcgatattcggaagaccacaaaactgaatatcggcggaaccgagattgaagtggaaacactggtggaagacgaggctgaggctggggctgtgaacaaggggactggcaaa

SEQ ID NO: 19 - protein, 315 aa, synthetic construct
  REGION 1..315: note = mutated Chop2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVICIHLSNLTGLSNDYSRRTMGLLVSDIGSIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 20 - protein, 315 aa, synthetic construct
  REGION 1..315: note = mutated Chop2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVIAIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 21 - DNA, 945 bp, synthetic construct
  misc_feature 1..945: note = mutated Chop2
  source 1..945: mol_type = other DNA
atggactacgggggggctctgtctgctgtcgggagggaactgctgtttgtgactaaccctgtcgtcgtgaacgggagtgtgctggtccctgaggaccagtgctactgtgccggctggatcgaatcacgcggaaccaacggggcccagacagctagcaatgtgctgcagtggctggccgctgggtttagtatcctgctgctgatgttctacgcctatcagacttggaagtcaacctgcggctgggaggaaatctacgtgtgcgctattgagatggtgaaagtgatcctggagttcttcttcgagttcaagaacccaagcatgctgtacctggctactggacaccgagtgcagtggctgagatatgcagaatggctgctgacatgccccgtcatcgccattcacctgtccaacctgacaggcctgagcaatgactactccaggagaactatgggactgctggtgtccgacatcggctgcattgtctggggagcaacttctgctatggcaaccggatacgtgaaggtcatctttttctgcctggggctgtgctatggcgcaaatacctttttccacgcagccaaggcctacattgaggggtatcataccgtgccaaaaggccggtgccgacaggtggtcacaggaatggcttggctgtttttcgtctcttggggaatgtttcccatcctgttcattctggggcctgaagggttcggcgtgctgtctgtctacggaagtacagtggggcatactatcattgacctgatgtccaaaaactgttggggcctgctgggacactatctgagagtgctgatccacgagcatatcctgattcatggcgatattcggaagaccacaaaactgaatatcggcggaaccgagattgaagtggaaacactggtggaagacgaggctgaggctggggctgtgaacaaggggactggcaaa

SEQ ID NO: 22 - protein, 315 aa, synthetic construct
  REGION 1..315: note = mutated Chop2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVIAIHLSNLTGLSNDYSRRTMGLLVSDIGCIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 23 - protein, 315 aa, synthetic construct
  REGION 1..315: note = mutated Chop2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGAIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 24 - DNA, 945 bp, synthetic construct
  misc_feature 1..945: note = mutated Chop2
  source 1..945: mol_type = other DNA
atggactacgggggggctctgtctgctgtcgggagggaactgctgtttgtgactaaccctgtcgtcgtgaacgggagtgtgctggtccctgaggaccagtgctactgtgccggctggatcgaatcacgcggaaccaacggggcccagacagctagcaatgtgctgcagtggctggccgctgggtttagtatcctgctgctgatgttctacgcctatcagacttggaagtcaacctgcggctgggaggaaatctacgtgtgcgctattgagatggtgaaagtgatcctggagttcttcttcgagttcaagaacccaagcatgctgtacctggctactggacaccgagtgcagtggctgagatatgcagaatggctgctgacatgccccgtcatctgcattcacctgtccaacctgacaggcctgagcaatgactactccaggagaactatgggactgctggtgtccgacatcggcgccattgtctggggagcaacttctgctatggcaaccggatacgtgaaggtcatctttttctgcctggggctgtgctatggcgcaaatacctttttccacgcagccaaggcctacattgaggggtatcataccgtgccaaaaggccggtgccgacaggtggtcacaggaatggcttggctgtttttcgtctcttggggaatgtttcccatcctgttcattctggggcctgaagggttcggcgtgctgtctgtctacggaagtacagtggggcatactatcattgacctgatgtccaaaaactgttggggcctgctgggacactatctgagagtgctgatccacgagcatatcctgattcatggcgatattcggaagaccacaaaactgaatatcggcggaaccgagattgaagtggaaacactggtggaagacgaggctgaggctggggctgtgaacaaggggactggcaaa

SEQ ID NO: 25 - protein, 315 aa, synthetic construct
  REGION 1..315: note = mutated Chop2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVICIHLSNLTGLSNDYSRRTMGLLVSDIGAIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 26 - protein, 315 aa, synthetic construct
  REGION 1..315: note = ChR2 mutant construct
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK